(19) United States
(12) Patent Application Publication
(10) Pub. No.: US 2007/0053925 A1
(43) Pub. Date: Mar. 8, 2007

(54) Staphylococcus aureus efb protein and c3 binding region which inhibit complement activation

(76) Inventors: Eric Brown, Houston, TX (US); Lawrence Lee, Houston, TX (US); Magnus Hook, Houston, TX (US)

Correspondence Address: STITES & HARBISON PLLC, 1199 NORTH FAIRFAX STREET, SUITE 900, ALEXANDRIA, VA, 22314, US

(21) Appl. No.: 10/553,225

(22) PCT Filed: Apr. 16, 2004
(86) PCT No.: PCT/US04/11949

§ 371 Date: Sep. 13, 2006

Related U.S. Application Data
(60) Provisional application No. 60/463,028, filed on Apr. 16, 2003.

(51) Int. Cl. A61K 39/02; G01N 33/554; C07K 14/31; C07K 16/12

(52) U.S. Cl. 424/190.1; 435/7.32; 530/350; 530/388.4

(57) ABSTRACT

The Efb protein from Staphylococcus aureus has now been shown to have the ability to bind to the C3 protein which is a crucial component in the activation of complement, and a specific C3 binding region has been located at the C-terminal end of the Efb protein. Isolated proteins and protein fragments containing the Efb protein C3 binding region are thus provided which have complement inhibiting activity, and these proteins and fragments are particularly useful in therapeutic methods wherein the inhibition of complement is desirable, such as in the treatment of hemolytic anemia, the prevention of graft or implant rejection, and to alleviate complement activation that is associated with kidney dialysis methods such as hemodialysis.

### CROSS REFERENCE TO RELATED APPLICATIONS

The present application claims the benefit of U.S. provisional application Ser. No. 60/463,028 filed Apr. 16, 2003.

### FIELD OF THE INVENTION

The present invention relates in general to certain fibrinogen binding proteins from Staphylococcus aureus and their ability to inhibit complement activation, and in particular to the 19 kDa extracellular fibrinogen binding protein (“SAC3” or “Efb”) from Staphylococcus aureus that has the ability to bind to the α-chain of the complement protein C3, and which includes a C3-binding region of Efb at the C-terminal end of the protein, which can be utilized so as to prevent complement activation in general and to be useful in a number of specific medical applications, such as the treatment of hemolytic anemia, wherein complement inhibition is highly desirable. Finally, the invention relates to isolated and purified complement-inhibiting forms of the Efb protein and/or its C3-binding region, as well as pharmaceutical compositions containing said proteins or said binding regions, nucleic acids coding for said proteins and/or said binding regions, antibodies recognizing said protein and/or said binding regions, and to therapeutically effective methods utilizing said proteins and/or said binding regions wherein the therapeutic benefit is achieved through the suppression of complement activation.

### BACKGROUND OF THE INVENTION

The successful, colonization of the host is a process required for most microorganisms to cause infections in animals and humans. Microbial adhesion is the first crucial step in a series of events that can eventually lead to disease. Pathogenic microorganisms colonize the host by attaching to host tissues or serum conditioned implanted biomaterials, such as catheters, artificial joints, and vascular grafts, through specific adhesins present on the surface of the bacteria. MSCRAMM®s (Microbial Surface Components Recognizing Adhesive Matrix Molecules) are a family of cell surface adhesins that recognize and specifically bind to distinct components in the host's extracellular matrix. Once the bacteria have successfully adhered and colonized host tissues, their physiology is dramatically altered and damaging components such as toxins and proteolytic enzymes are secreted. Moreover, adherent bacteria often produce a biofilm and quickly become more resistant to the killing effect of most antibiotics.

One of the most common bacterial organisms which is responsible for a variety of infections and other health problems is Staphylococcus aureus (S. aureus or “SA”). While S. aureus can colonize the skin and anterior nares in up to about 20% of humans without causing disease or discernable clinical symptoms (1, 2), SA also remains one of the world's primary health threats, and indeed the emergence of antibiotic-resistant SA strains has posed severe health threats in hospitals and communities (1, 3). Furthermore, the broad spectrum of diseases that can result from SA infections (e.g. skin infections, arthritis, or septic shock) is a reflection of this organism's capacity to not only colonize a variety of different tissues via different MSCRAMM®-ligand interactions (4-14) but to also circumvent a variety of immune surveillance systems which result in the persistence of SA in radically different environments within the host organism (15-19).

Because tissue colonization is a critical first step in any infection process and pathogens associated with persistent infections must constantly avoid host immunity to survive, it is not surprising that some MSCRAMM®s can also serve as MIMs (Microbial Immunomodulatory Molecules) (18). For example, the secreted SA Map (MHC Analog Protein) not only adheres to a variety of extracellular matrix (ECM) components (20, 21) but can also negatively affect T cell immunity and prevent neutrophil migration (15, 18), and the Streptococcal M protein which binds fibrinogen (Fgn) can also interfere with complement activabon (alternative pathway) by mechanisms not clearly defined (22).

The complement system represents the most ‘primitive’ line of defense against infectious agents. Complement systems exist in all vertebrates (23) and complement-component analogs can be found in some nonvertebrates (e.g. horseshoe crab and insects) (23). Evolutionarily, the complement system at many levels became associated with humoral immunity, and it now functions as a primary antibody effector mechanism (23). The importance of the complement system as a primary and first line of defense mechanism in vertebrates is mirrored by the fact that some infectious agents including bacteria, viruses, protozoans, helminthes, and fungi, have developed survival strategies which interfere with, avoid, or manipulate complement system components (23-26). Although the complement system is comprised of at least 20 immunologically and chemically distinct proteins, the effector functions of the complement cascade are carried out by the components designated C1-C9 (27). One of the most critical components of the complement cascades is the C3 protein which is the common link between the alternative, classical and mannose-binding lectin (MBL) pathways of complement activation (27).

In a mouse model of SA-induced septicemia and septic arthritis, mice that had been depleted of complement (using cobra venom factor) exhibited significantly increased disease severity after SA induction, suggesting a role for complement in controlling haematogenously-acquired SA infections (28). Conversely, intradermally-infected, C3-deficient mice were more resistant to SA infections compared to wild-type control mice, suggesting the possibility that SA may actively interact with C3 (29, 30). In any event, no SA immunoregulatory proteins with complement binding activity have been previously described to date, and thus it remains an important object of identify and isolate such proteins, including any regions or regions therein which exhibit complement binding activity, and to utilize such isolated and/or purified proteins or binding regions in methods of treating or preventing infections wherein the bacteria may be secreting proteins which bind to complement and thus disrupt host complement activity. In addition, to the extent such proteins or binding regions could be isolated, they would represent an important means to provide complement-inhibiting activity in the wide variety of medical procedures wherein such activity is highly desirable.

### SUMMARY OF THE INVENTION

Accordingly, it is an object of the present invention to provide isolated and/or purified proteins or binding regions from staphylococcus aureus which can bind to the C3 protein and thus be useful in methods wherein complement inhibition is desired.

It is further an object of the present invention to provide an isolated and/or purified Efb protein from Staphylococcus aureus, and to provide an isolated and/or purified C3 binding region from said protein, which can disrupt the complement pathways and be useful in methods of achieving suppression of complement activity when such suppression is considered desirable.

It is still further an object of the present invention to provide pharmaceutical compositions which can be used to effect complement inhibition in a human or animal patient which are comprised of an isolated and/or purified Efb protein or an isolated and/or purified protein fragment containing the C3 binding region from the Efb protein in an amount effective to achieve complement inhibition combined with a pharmaceutically acceptable vehicle, carrier or excipient.

It is another object of the present invention to provide antibodies which can recognize or bind to the Efb protein, or which can recognize or bind to the C3 binding region of Efb or protein fragments containing the Efb C3 binding region, and which can be useful in methods of diagnosing, treating or preventing a staphylococcal infection.

It is yet another object of the present invention to provide nucleic acids which encode the Efb protein and which encode the C3 binding region of the Efb protein and/or fragments containing said C3 binding region.

It is yet another object of the present invention to provide passive or active vaccines which utilize the Efb protein or its C3 binding region, or antibodies thereto, and to provide methods of generating and/or isolating antibodies or antisera from the Efb protein, its C3 binding region, and/or fragments containing said C3 binding region.

It is yet a further object of the invention to provide methods of diagnosing, treating or preventing staphylococcal infection using Efb protein and/or its C3 binding region and/or antibodies thereto, and to provide methods of achieving complement inhibition in a human or animal patient using a complement-inhibiting effective amount of the Efb protein or its C3 binding region, or fragments containing said C3 binding region, so as to treat specific conditions such as hemolytc anemia, or to prevent unwanted deleterious side effects associated with graft rejection, xenogeneic transplanted tissues, and kidney dialysis, in particular hemodialysis.

These and other objects are provided by virtue of the present invention which is based on the discovery for the first time of an SA protein which can bind to the SA complement protein C3 and which can thus be used to disrupt any of a number of classical and alternative complement activation pathways which may occur in a host human or animal. This protein; which has been referred to as the Efb protein, or “SAC3” (S. aureus C3-binding protein) is a 19 kDa constitutively secreted protein that was previously only known to bind Fgn and was thought to interfere with platelet aggregation so as to play a role in delaying wound healing (34). The present invention thus provides for the first time an isolated SA protein and a binding region isolated from said protein which can bind the C3 protein and which can thus be utilized in therapeutic methods wherein inactivation of the complement pathways and inhibition of complement activity must be achieved to obtain the therapeutic benefit, including procedures relating to treating hemolytic anemia, suppression of graft rejection, such as in the case of tissue implants or xenogeneic transplanted tissues or organs, and alleviating undesirable deleterious effects associated with complement activity arising during kidney dialysis procedures such as hemodialysis.

The present invention also relates to the generation of antibodies from the Efb protein and/or its C3 binding region and their use in diagnosing, treating or preventing staphylococcal infections, along with therapeutic pharmaceutical compositions made from the isolated proteins or C3 binding regions or antibodies thereto which are useful in therapeutic procedures wherein complement inhibition is desired. In addition, nucleic acids encoding the Efb protein and/or its C3 binding region or fragments containing the Efb binding region, are also encompassed by the present invention.

These embodiments and other alternatives and modifications within the spirit and scope of the disclosed invention will become readily apparent to those skilled in the art from reading the present specification and/or the references cited herein, all of which are incorporated by reference.

### BRIEF DESCRIPTION OF THE DRAWING FIGURES

FIG. 1a and 1b are chromatographic depictions of the secretion of the SA C3b-binding protein of the present invention wherein Lane 1 is E. coli; lane 2 is S. carnosus; lane 3 is S. aureus; and lane 4 is S. aureus. supernatant. Bacteria or supernatant were fractionated by SDS-PAGE (a) or transferred onto a PVDF membrane (b). After blocking additional protein-binding sites, proteins on the membranes were probed with digoxigenin-labeled, C3b followed by a secondary incubation with AP-labeled anti-digoxigenin Fab fragments and then developed. Membranes probed with secondary antibody alone did not exhibit nonspecific binding (data not shown).

FIGS. 2a-2c are chromatographic depictions showing labeled C3b and Fgn bind to a recombinant SAC3 (Efb) protein in accordance with the present invention. In the figure, Lane 1 is SAC3; lane 2 is SA1755; lane 3 is Map19; and lane 4 is SdrG. 4 μg of each recombinant protein was subjected to SDS-PAGE (a) or transferred onto PVDF membranes. After blocking additional protein-binding sites, proteins on the membranes were probed with either digoxigenin-labeled C3b (b) or Fgn (c) followed by a secondary incubation With AP-labeled anti-digoxigenin Fab fragments and then developed. Membranes probed with secondary antibody alone did not exhibit nonspecific binding (data not shown).

FIGS. 3a-3c are chromatographic depictions showing the SAC3 (Efb) protein of the invention binds to membrane-bound C3b. Lane 1 is C3b; lane 2 is human Fgn; lane 3 is human serum. Proteins and serum were subjected to SDS-PAGE (a) or transferred onto PVDF membranes (b-c). After blocking additional protein-binding sites, proteins on the membranes were probed with either SAC3 (b) or biotin-labeled chicken anti-human C3 antibodies (c) followed by a secondary incubation with either mouse anti-His antibodies (b) or with avidin-AP (c). Following a third incubation with anti-mouse AP-conjugated antibodies. (b) the blots were developed. Neither secondary AP-conjugated secondary antibodies alone nor avidin-AP alone bound nonspecifically to the membranes (data not shown).

FIGS. 4a-4b are graphic representations of SAC3 (Efb) binding to C3b- and Fgn-coated microtiter wells. SAC3 or SdrG were used to probe C3b- or Fgn-coated microtiter wells (a and b, respectively). Binding was detected using a primary monoclonal mouse anti-His followed by a secondary incubation with a goat anti-mouse AP-conjugated antibody. The data are expressed as the mean absorbance (405 nm) ±SE of the mean of triplicate samples.

FIGS. 5a-5b are graphic representation of the inhibition of complement activation by the SA SAC3 (Efb) protein in accordance with the present invention. The effects of SAC3 on complement activation were examined using different assays measuring the classical (a) or alternative (b) complement activation pathways. Preincubation of human serum with SAC3 but not with control proteins prevented the complement-mediated lysis of erythrocytes. These experiments were performed 3 times and the data are representative of all results.

FIG. 6 is a schematic representation of the structural organization of the SAC3 (Efb) protein in accordance with the present invention wherein S is the signal sequence; R is the N-terminal region of the protein that contains two homologous repeat regions and is known to, bind to fibrinogen; U is the unique region which is believed to contain a binding site for solid-phase fibrinogen but not soluble fibrinogen. rEfb, full length Efb protein without the signal sequence; rEfb120, contains the R-region and 23 amino acids of the U-region of the protein; and rEfb165, contains the U-region. All recombinant proteins are expressed with N-terminal 6× His tags.

FIG. 7 is a chromatographic representation showing that the Efb C-terminal region in accordance with the present invention Efb binds to the C3 protein. In the figure, Lane 1 is rEfb; Lane 2 is rEfb120; and Lane 3 is rEfb165, these proteins and regions were subjected to SDS-PAGE and A, Coomassie-stained or B, transferred to PVDF for western-ligand analysis and probed with 5 μg of digoxigenin-labeled C3b or C, digoxigenin-labeled fibrinogen. Bound proteins were detected using AP conjugated anti-digoxigenin Fab fragments (1:15,000) and then developed.

FIGS. 8A-B are graphic representations showing an ELISA of rEfb constructs for plate-bound C3 and fibrinogen. Microtiter wells were coated with 0.25 μg of A, C3b or B, fibrinogen. rEfb (open squares), rEfb120 (open circles), or rEfb165 (closed circles) were used at various concentrations to determine their binding specificity for C3b or fibrinogen. The Efb proteins were detected using anti-His antibodies (1:5,000) followed by a secondary antibody conjugated to AP (1:5,000) and then developed. The data are expressed as the mean absorbance (405 nm) ±SE of the mean of triplicate samples.

FIG. 9 is a bar graph showing that the C-terminal region of Efb (rEfb165) in accordance with the present invention inhibits complement-mediated RBC lysis. The effects of rEfb, rEfb120, and rEfb165 on complement activation were examined using an assay to measure the classical complement activation pathway. The data are expressed as percent RBC lysis of the, complement standard reference serum.

FIGS. 10A-B are chromatographic representations showing that rEfb binds to the C3d fragment of C3. Lane 1, C3b; Lane 2, iC3b; Lane 3, C3c; Lane 4, C3d were subjected to SDS-PAGE and A, Coomassie-stained or B, transferred to PVDF for western-ligand analysis and probed with 10 μg of rEfb. rEfb was detected using anti-His antibodies (1:15,000) followed by a secondary antibody conjugated to AP (1:15,000) and then developed.

FIGS. 11A-B are bar graphs showing that the multiple binding regions of rEfb allow for simultaneous binding to C3 and fibrinogen. Microtiter wells were coated with 0.25 μg of fibrinogen, probed with 1 μg of rEfb, rEfb120, or rEfb165 and then probed with 0.25 μg of digoxigenin-labeled C3b. A, digoxigenin-labeled C3b were detected using AP-conjugated anti-digoxigenin (Fab fragment) (1:5000) and then developed. B, rEfb proteins bound to fibrinogen was detected using ant-His antibodies (1:5,000) followed by a secondary antibody conjugated to AP (1:5,000) and then developed. The data are expressed as the mean absorbance (405 nm) ±SE of the mean of triplicate samples.

### DETAILED DESCRIPTION OF THE PREFERRED EMBODIMENTS

In accordance with the present invention, the present inventors have surprisingly discovered that the Efb protein of staphylococcus aureus has the ability to bind to the C3 protein which is crucial in the various pathways of complement activation, and that the Efb protein has a C3 binding region at the C-terminal end of this protein. The Efb protein, also referred to as the “SAC3” protein, is as 19 kDa protein from Staphylococcus aureus that has previously been observed to have fibrinogen binding activity. Further information about the Efb protein, including amino acid and nucleic acid sequence information, is disclosed in patent references including U.S. Pat. No. 6,299,879, U.S. Pat. App. Pub. No. 2002/0173462, European Patent Application 621875A1, European Patent 621875B,1, and PCT Publication WO 94/06830, and other members of this patent family. All of said patent references are incorporated herein by reference. This protein is further described in journal articles, including Bodén et al, Infect Immun. 57, 2358-63 (1989), Bodén et al., Microb. Pathog. 12, 289-98 (1992), and Bodén et al., Mol. Microbiol. 12, 599-606 (1 994), said articles incorporated herein by reference. Finally, the Efb protein has been sequenced and is included in GenBank, such as in accession numbers X72014 and X72015, and in AJ306909, and these sequences are incorporated herein by reference as well.

In accordance with the invention, the unexpected ability of Efb to bind the C3 protein and to block opsonophagocytosis can be utilized in therapeutic methods wherein complement inhibition is the desired object, as will be described further below. Accordingly, in accordance with the invention comprises compositions containing the Efb protein in an amount effective to inhibit complement activation, and further to antibodies, vaccines and methods which utilize this protein, as well as to the nucleic acids that code for this protein.

An additional surprising and unexpected discovery was that Efb has a specific C3 binding region which accounted for the C3 binding ability and the ability to disrupt all of the known complement pathways, and that this C3 binding region was located at the C-terminal region of the Efb protein. Accordingly, in accordance with the present invention, the isolated and/or purified C3 binding region, as well as isolated and/or purified protein fragments containing this binding region in its active form, are also useful in the present invention to inhibit complement activation and thus be useful in therapeutic methods wherein it is desired to inhibit complement activation. In addition, the invention also contemplates compositions containing the C3 binding region, and/or protein fragments containing this binding region, in an amount effective to inhibit complement activation, and further to antibodies, vaccines and methods which utilize the C3 binding region, as well as to the nucleic acids that code for this binding region.

As will be recognizable by one of ordinary skill in this art, the Efb protein and its C3 binding region and fragments containing this region can be isolated in any suitable manner well known in the art. For example, the Efb protein can be identified and isolated by the use of conventional fractionation techniques, and in one such suitable method, S. aureus bacteria are grown in suitable nutrient broth while shaking and then are washed using suitable saline solutions such as phosphate buffered saline (PBS, pH 7.4). Bacteria obtained in the SA supernatant from such cultures can be fractionated such as by SDS-PAGE and examined by staining with a suitable stain such as 0.05% Coomassie brilliant blue R250, or subjected to electro-transfer to a suitable membrane (e.g., polyvinylidene fluoride) so as to identify and isolate the 19 kDa Efb protein as described previously (35). In order to examine the C3 binding properties, labeled C3 or C3b (Advanced Research Technologies, San Diego, Calif., USA) can be introduced to the isolated proteins, such as through Western-ligand blotting, and binding was visualized by incubation with suitable binding labels The characterization of the 19 kDa C3-binding protein can further be accomplished through additional fractionation and staining steps as would be understood by one skilled in the art.

Similarly, one skilled in the art will also be able to obtain the C3-binding region from the Efb protein using conventional methods well understood in the art. In accordance with the invention, the isolation and identification of the C3 minimum binding region was accomplished using truncations from the nucleic acid encoding the Efb protein which were amplified using PCR techniques and then expressed and tested for their C3-binding ability. The particular truncations were used to express recombinant proteins using standard E. coli expression vectors such as the pCRT7/NT-TOPO (Invitrogen) expression vector in E. coli (BL21) harboring the corresponding plasmids, respectively, and the proteins or protein fragments were expressed and purified using readily available conventional methods. These obtained proteins and fragments were then tested to determine their ability to bind the C3 protein or its related proteins in the complement pathway and their ability to affect complement activity. As indicated herein, the C3 binding regions of the present invention were located at the C-terminal region of the Efb protein and are characterized by their ability to bind C3 or its related proteins in the complement activation pathway so as to interfere with complement activation. Using these techniques, it was determine that the C3 binding region was identified as the recombinant protein rEfb165 from the C-terminal end of the Efb protein, which could inhibit complement-mediated lysis of RBCs to the same degree as the full Efb protein, and this binding region was contrasted with recombinant protein rEfb120 taken from the other end of the protein which exhibited no such inhibitory activity.

In general, the Efb proteins and C3 binding regions of the invention can thus be prepared using the well known techniques of solid phase, liquid phase, or peptide condensation techniques, or any combination thereof, and can include natural and unnatural amino acids. Amino acids used for peptide synthesis may be standard Boc (N^{a}-amino protected N^{a}-t-butyloxycarbonyl) amino acid resin with the standard deprotecting, neutralization, coupling and wash protocols of the original solid phase procedure of Merrifield (J. Am. Chem. Soc., 85:2149-2154, 1963), or the base-labile N^{a}-amino protected 9-fluorenylmethoxycarbonyl (Fmoc) amino acids first described by Carpino and Han (J. Org. Chem., 37:3403-3409, 1972). Both Fmoc and Boc N^{a}-amino protected amino acids can be obtained from Fluka, Bachem, Advanced Chemtech, Sigma, Cambridge Research Biochemical, Bachem, or Peninsula Labs or other chemical companies familiar to those who practice this art. In addition, the method of the invention can be used with other N^{a}-protecting groups that are familiar to those skilled in this art. Solid phase peptide synthesis may be accomplished by techniques familiar to those in the art and provided, for example, in Stewart and Young, 1984, Solid Phase Synthesis, Second Edition, Pierce Chemical Co., Rockford, Ill.; Fields and Noble, 1990, Int. J. Pept Protein Res. 35:161-214, or using automated synthesizers, such as sold by ABS. Thus, the proteins and binding regions of the invention may comprise D-amino acids, a combination of D- and L-amino acids, and various. “designer” amino acids (e.g., β-methyl amino acids, Cα-methyl amino acids, and Nα-methyl amino acids, etc.) to convey special properties, in all cases retaining the C3 binding ability of the Efb protein and the. C3 binding region. Synthetic amino acids include ornithine for lysine, fluorophenylalanine for phenylalanine, and norleucine for leucine or isoleucine. Additionally, by assigning specific amino acids at specific coupling steps, α-helices, β turns, β sheets, γ-turns, and cyclic peptides can, be generated.

Accordingly, in accordance with the invention, there is provided isolated and/or purified Efb proteins from Staphylococcus aureus which can bind to the C3 protein and thus be useful in methods wherein complement inhibition is desired, and in addition, there is provided an isolated and/or purified C3 binding region from the Efb protein which is that minimum binding region necessary to be able to bind the C3 protein or other proteins in the complement activation pathways, and which can inhibit complement activation and the complement-mediated lysis of red blood cells. By C3 binding region is thus meant to include that region of the Efb protein which includes the minimum binding region necessary to bind the C3 protein and inhibit complement activity, and thus active fragments containing this minimum binding region which also inhibit complement activity are included in this term. In accordance with the invention, isolated and/or purified Efb proteins and/or an isolated and/or purified C3 binding region from said protein, are provided which, can disrupt the complement pathways and be useful in methods of achieving suppression of complement activity when such suppression is considered desirable, such as described further below

As indicated above, the invention relates to the Efb protein and C3 binding region of S. aureus, and this includes the amino acid sequences coding for these proteins and regions as well as the nucleic acid sequences coding for these proteins and regions. However, as would be recognized by one of ordinary skill in this art, modification and changes may be made in the structure of the proteins and C3 binding regions of the present invention as well as to the nucleic acid segments, which encode them and still obtain a functional molecule that encodes a protein, fragment or peptide with the desirable C3 binding characteristics. The amino acid changes may be achieved recombinantly by changing the codons of the DNA sequence. For example, certain amino acids may be substituted for other amino acids in a protein structure without appreciable loss of C3 binding ability as would be understood by one skilled in the art. For example, since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the protein and peptide sequences coding Efb and the C3 binding region, or to the corresponding DNA sequences which encode said proteins or regions without appreciable loss,of their biological utility or activity.

Accordingly, acceptable amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine.

Moreover, it is understood that nucleic acids coding for the amino acid sequences of the present invention are also subject to modification in that there are typically several different codons that encode the same amino acids. Accordingly, the nucleic acids coding for the Efb protein and C3 binding region of the present invention include numerous sequences which all code for the same amino acid sequence.

Pharmaceutical Compositions of Efb and C3 Binding Region

In accordance with the present invention, there are provided pharmaceutical and therapeutic compositions containing the Efb protein or C3 binding region, may be formulated in combination with a pharmaceutical vehicle, excipient or carrier such as saline, dextrose, water, glycerol, ethanol, other therapeutic compounds, and combinations thereof. The formulation should be appropriate for the mode of administration. These compositions will preferably contain the Efb protein of the C3 binding region in an amount effective to achieve an inhibition of complement activation in a human or animal patient. For administration of the Efb protein, C3 binding region, or pharmaceutically acceptable compositions containing these agents in accordance with the invention, an “effective amount” is that amount that would be readily determinable by one of ordinary skill in the art to effectively treat a patient or to inhibit complement activation in a patient, and that amount would be determined by the specific circumstances and conditions surrounding the mode of therapy, such as nature of patient and condition to be treated, nature of the materials used in the composition, desired purpose for the treatment, etc. As indicated above, these compositions will be extremely useful in therapeutic methods wherein the inhibition of complement activation is highly desirable, including the treatment of hemolytic anemia, prevention of graft, tissue or organ rejection from transplanted materials, and the suppression of complement activation during kidney dialysis operations, in particular hemodialysis. They may also be useful in treating other completed-mediated disorders such as systemic lupus erythematosus and autoimmune arthritis

Suitable methods of administration of any pharmaceutical composition disclosed in this application include, but are not limited to, topical, oral, anal, vaginal, intravenous, intraperitoneal, intramuscular, subcutaneous, intranasal and intradermal administration.

For topical administration, the composition is formulated in the form of an ointment, cream, gel, lotion, drops (such as eye drops and ear drops), or solution (such as mouthwash). Wound or surgical dressings, sutures and aerosols may be impregnated with the composition. The composition may contain conventional additives, such as preservatives, solvents to promote penetration, and emollients. Topical formulations may also contain conventional carriers such as cream or ointment bases, ethanol, or oleyl alcohol.

Antibodies and Vaccines

In accordance with the present invention, there are also provided antibodies which can recognize and bind to the Efb protein and to its C3 binding region.

In addition to Efb protein and the C3 binding region as identified and isolated above, the present invention contemplates the generation of antibodies which recognize and which can bind to the Efb protein and/or its C3 binding region. By “antibody” is meant any intact antibody molecule or fragments thereof that recognize antigen (e.g. Fab or F(ab′)2 fragments) and can be of polyclonal or monoclonal type, and the antibodies in accordance with the invention will be capable of recognizing the Efb protein and/or its C3 binding region. These antibodies thus produced may be utilized in methods of diagnosing, monitoring, treating or preventing infection from Staphylococcus aureus in that they can be used to disrupt the C3 binding process in situations wherein such disruption is desirable, such as in the interest of treating or, preventing the spread of SA infection. These antibodies may be monoclonal or polyclonal and may be generated using any suitable method to raise such antibodies such as would be well known in this art.

For example, with regard to polyclonal antibodies, these may be generated using a number of suitable methods generally involving the injection of the isolated and/or purified or recombinantly produced Efb proteins or C3 binding regions (or their immunogenic active peptides or epitopes) into a suitable host in order to generate the polyclonal antibodies which can then be recovered from the host. For example, in accordance with the invention, an isolated and/or purified Efb proteins or C3 binding regions may be injected into rabbits in order to generate polyclonal antibodies and antisera which recognize them. In addition, monoclonal antibodies in accordance with the invention may be generated using a suitable hybridoma as would be readily understood by those of ordinary skill in the art, including the well known method of Kohler and Milstein, Nature 256.495-497 (1975), or other suitable ways known in the field, such as those methods disclosed in U.S. Pat. Nos. 6,331,415; 5,981,216; 5,807,715; and 4,816,567; Eur. Pat. App. 519,596; and PCT publication WO 00/71585, all of these patent publications incorporated herein by reference. These methods include their preparation as chimeric, humanized, or human monoclonal antibodies in ways that would be well known in this field. Still further, monoclonal antibodies may be prepared from a single chain, such as the light or heavy chains, and in addition may be prepared from active fragments of an antibody which retain the binding characteristics (e.g., specificity and/or affinity) of the whole antibody. By active fragments is meant an antibody fragment which has the same binding specificity as a complete antibody which binds to Efb protein or C3 binding region, or its homologue from the different staph bacteria, and the term “antibody” as used herein is meant to include said fragments. Additionally, antisera prepared using monoclonal or polyclonal antibodies in accordance with the invention are also contemplated and may be prepared in a number of suitable ways as would be recognized by one skilled in the art.

In accordance with the present invention, there are provided pharmaceutical and therapeutic compositions containing antibodies which recognize the Efb protein or its C3 binding region, and these compositions may be formulated in combination with a pharmaceutical vehicle, excipient or carrier such as saline, dextrose, water, glycerol, ethanol, other therapeutic compounds, and combinations thereof. The formulation should be appropriate for the mode of administration. Suitable methods of administration of any pharmaceutical composition disclosed in this application include, but are not limited to, topical, oral, anal, vaginal, intravenous, intraperitoneal, intramuscular, subcutaneous, intranasal and intradermal administration. For topical administration, the composition is formulated in the form of an ointment, cream, gel, lotion, drops (such as eye drops and ear drops), or solution (such as mouthwash). Wound or surgical dressings, sutures and aerosols may be impregnated with the composition. The composition may contain conventional additives, such as preservatives, solvents to promote penetration, and emollients. Topical, formulations may also contain conventional carriers such as cream or ointment bases, ethanol, or oleyl alcohol.

The antibody compositions of the present invention may also be administered with a suitable adjuvant in an amount effective to enhance the immunogenic response. For example, suitable adjuvants may include alum (aluminum phosphate or aluminum hydroxide), which is used widely in humans, and other adjuvants such as saponin and its purified component Quil A, Freund's complete adjuvant, RIBBI adjuvant, and other adjuvants used in research and veterinary applications. Still other chemically defined preparations such as muramyl dipeptide, monophosphoryl lipid A, phospholipid conjugates such as those described by Goodman-Snitkoff et al. J. Immunol. 147:410-415 (1991) and incorporated by reference herein, encapsulation of the conjugate within a proteoliposome as described by Miller et al., J. Exp. Med. 176:1739-1744 (1992) and incorporated by reference herein, and encapsulation of the protein in lipid vesicles such as Novasome™ lipid vesicles (Micro Vescular Systems, Inc., Nashua, NH) may also be useful.

In any event, the antibody compositions of the present invention which recognize the Efb protein and/or its C3 binding region will be useful in methods of preventing or treating staphylococcal infection. In accordance with the present invention, methods are provided for preventing or treating a staphylococcal infection are thus provided which comprise administering an effective amount of an antibody as set forth herein so as to treat or prevent a staphylococcal infection.

In addition to use in methods or treating or preventing a staphylococcal infection, the antibodies of the invention may also be used for the specific detection of staphylococcal proteins, or as research tools. The term “antibodies” as used herein includes monoclonal, polyclonal, chimeric, single chain, bispecific, simianized, and humanized or primatized antibodies as well as Fab fragments, such as those fragments which maintain the binding specificity of the antibodies to the surface proteins specified above, including the products of an Fab immunoglobulin expression library. Accordingly, the invention contemplates the use of single chains such as the variable heavy and light chains of the antibodies. Generation of any of these types of antibodies or antibody fragments is well known to those skilled in the art. In the present case, antibodies to the surface proteins or their active regions as referred to above can be generated, isolated and/or purified, and then used to treat or protect against staphylococcal infection.

The compositions of the present invention, including the pharmaceutical compositions and antibody compositions as set forth above, may also be utilized in the development of vaccines for active and passive immunization against staphylococcal infections. In the case of active vaccines, said vaccines are prepared by providing an immunogenic amount of the Efb protein or the C3 binding region of, the present invention along with a pharmaceutically acceptable vehicle, carrier or excipient and may also include adjuvants or carriers as discussed above which enhance immunogenicity. As referred to above, an “immunogenic amount” of the antigen to be used in accordance with the invention is intended to mean a nontoxic but sufficient amount of the agent, such that an immunogenic response will be elicited in the host so that the desired prophylactic or therapeutic effect is produced. Accordingly, the exact amount of the antigen that is required will vary from subject to subject, depending on the species, age, and general condition of the subject, the severity of the condition being treated, the particular carrier or adjuvant being used and its mode of administration, and the like. Similarly, the “immunogenic amount” of any such antigenic vaccine composition will vary based on the particular circumstances, and an appropriate immunogenic amount may be determined in each case of application by one of ordinary skill in the art using only routine experimentation. The dose should be adjusted to suit the individual to whom the composition is administered and will vary with age, weight and metabolism of the individual.

In the case of passive vaccines, it is contemplated that an effective amount of an antibody in accordance with the invention will be useful as a passive vaccine, and this effective amount of an antibody is that amount effective in treating or preventing a staphylococcal infection. Such a passive vaccine can also comprise a suitable, pharmaceutically acceptable vehicle, excipient or carrier which will be those known and commonly used in the vaccine arts.

Methods:

Methods of Inhibiting Complement Activation

In accordance with the present invention, there is provided a method of inhibiting complement activation, i.e., preventing the activation of any of the complement pathways and inhibiting complement-mediated lysis of red blood cells (RBC's) which comprises administering to a patient in need of such therapy and effective amount of an Efb protein or a C3 binding region as described further above. By effective amount is meant that amount that would be readily determinable by one of ordinary skill in the art to effectively treat a patient or to inhibit complement activation in a patient, and that amount would be determined by the specific circumstances and conditions surrounding the mode of therapy, such as nature of patient and condition to be treated, nature of the materials used in the composition, desired purpose for the treatment, etc. As indicated above, these compositions will be extremely useful in therapeutic methods wherein the inhibition of complement activation is highly desirable, including the treatment of hemolytic anemia, prevention of graft, tissue or organ rejection from transplanted materials, and the suppression of complement activation during kidney dialysis operations, in particular hemodialysis. Accordingly, the present invention provides methods of treating such diseases or conditions, e.g., hemolytic anemia, which comprises administering to the patient an Efb protein from S. aureus or the C3 binding region from the S. aureus Efb protein in an amount effective to inhibit complemerit activation, i.e., inhibit complement-mediated lysis of RBC's. These methods may also employ therapeutic pharmaceutical compositions of the Efb protein or C3 binding region wherein an effective amount of these proteins or regions is used along with a pharmaceutically acceptable vehicle, excipient or carrier.

As indicated above, the methods of the present invention include any therapeutic method wherein inhibition of complement activation is the desired goal. In addition to hemolytic anemia, it is also contemplated that the present method will be useful in inhibiting or reducing graft or implant rejection such as when a biological or artificial prosthetic implant is transplanted into a human or animal patient. This will particularly be the case in tissue and organ transplants, either from human or non-human sources, such as in xenotransplantation, wherein suppression of the complement pathways is important to prevent tissue or organ rejection, especially complement-mediated hyperacute rejection. In addition, there may be circumstances wherein during such a method, it might be advantageous to coat medical devices or polymeric biomaterials with the Efb or C3 binding regions of the present invention to further enhance the inhibition of complement activation. It will be understood in all of these cases that the application or administration of the Efb protein or C3 binding region will be at a non-toxic amount effective to provide inhibition of complement as needed for a given procedure and patient. Accordingly, the preferred dose for administration of the proteins, binding regions and compositions of the invention will vary greatly depending on the nature of the condition treated and the nature and condition of a patient, and will generally be that non-toxic amount suitable to achieve the desired therapeutic benefit obtained by inhibition of complement in a given case.

Eliciting an Immune Response

In accordance with the present invention, a method is provided for eliciting an immunogenic reaction in a human or animal comprising administering to the human or animal an immunologically effective amount of the Efb protein of the C3 binding region in accordance with the invention. Such a method will involve administering to a human or animal an immunogenic amount of the Efb protein or C3 binding region in accordance with the invention so as to elicit an immunogenic response. As indicated above, an “immunogenic amount” is intended to mean a nontoxic but sufficient amount of the agent, such that an immunogenic response will be elicited in the host so that the desired prophylactic or therapeutic effect is produced. Accordingly, the exact amount of the isolated protein that is required to elicit such a response will vary from subject to subject, depending on the species, age, and general condition of the subject, the severity of the condition being treated, the particular carrier or adjuvant being used and it s mode of administration, and the like. The invention also contemplates methods of generating antibodies, and suitable methods of generating monoclonal and polyclonal antibodies are described in more detail above.

Detecting and Diagnosing Infections

In accordance with the present invention, methods are provided, for identifying and diagnosing infections using the Efb protein and the C3 binding region. These methods are based on techniques well known in the art and can take generally two forms. In the first form, an Efb protein or C3 binding region in accordance with the invention is isolated, and a sample of a biological fluid which might contain antibodies to the protein or binding region is placed in contact with the protein or binding region. The presence of the antibodies is shown by the binding of the Efb protein or C3 binding region to the antibodies in the sample. Similarly, an alternative diagnostic method involves use of an antibody recognizing the Efb protein or the C3 binding region in accordance with the present invention. In such a case, the sample which might contain the Efb protein or C3 binding region is introduced to the antibodies of the invention, and the presence of binding by the antibody will indicate the presence of the Efb protein or the C3 binding region in the sample.

These methods would conventionally involve the use of detectable labels linked to the antibody or antigen as would be understood by one skilled in the art. The methods could be carried out using diagnostic kits well known in the art and will generally be prepared so as to be suitable for determining the presence of either the relevant antibody or antigens as described above in accordance with the invention. These diagnostic kits will generally include the antibodies or antigens of the invention along with suitable means for detecting binding by antibodies to the target antigen or the antigen to the target antibody so as to obtain a diagnosis based in the presence of the antigens or antibodies as described above.

Treating or Protecting Against Infections

In accordance with the present invention, methods are provided for preventing or treating an staphylococcal infection which comprise administering an effective amount of the antibodies as described above to a human or animal patient in need of such treatment in amounts effective to treat or prevent the infection. Accordingly, in accordance with the invention, administration of an effective amount of the antibodies of the present invention in any of the conventional ways described above (e.g., topical, parenteral, intramuscular, etc.), can provide,a useful method of treating or preventing staphylococcal infections in human or animal patients. In this case, effective amounts used in the treatment or prevention of infection is meant that level of use, such as of an antibody titer, that will be sufficient to treat or prevent a staphylococcal infection such as from Staphylococcus aureus.

Accordingly, the present invention provides compositions and methods which are primarily useful in therapeutic methods which require the inhibition of complement in order to give or improve the particular therapeutic benefit sought to be achieved.

### EXAMPLES

The following examples are provided which exemplify aspects of the preferred embodiments of the present invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Isolation and Identification of the SAC3 (Efb) Protein and Confirmation of its Complement Inhibiting Activity

Overview

We have identified a 19 kDa protein secreted by SA that can bind to the complement protein C3 and have designated this protein SAC3 (S. aureus C3-binding protein). N-terminal sequencing of SAC3 identified this protein as the SA extracellular fibrinogen-binding protein (Efb) (31-33). Efb is a constitutively secreted protein that not only binds Fgn, but can interfere with platelet aggregation and is hypothesized to play a role in delaying wound healing (34). The data presented in this report show that SAC3 is a MIM that may be involved in SA survival and persistence.

Materials and Methods

Identification of SAC3. Escherichia coli strain JM101, Staphylococcus carnosus strain TM300, and Staphylococcus aureus strain Newman were grown under shaking conditions overnight at 37° C in 5 ml Lennox broth (Sigma-Aldrich, St. Louis, Mo., USA) as described previously (18, 35). Bacteria were washed in phosphate buffered saline (PBS, pH 7.4) and total protein was quantified using the bicinchoninic acid protein assay (BCA) (Pierce Chemical Co., Rockford, Ill., USA). Bacteria (20 μg) or 20 μl of SA supernatant from an overnight culture were fractionated by SDS-PAGE (12% gel) under reducing conditions and examined by staining with 0.05% Coomassie brilliant blue R250 or subjected to electro-transfer to a 0.45 μm Immobilon-P™ PVDF (polyvinylidene fluoride) membrane (Millipore, Bedford, Mass., USA) as described previously (35). Western-ligand blots were performed by blocking the membranes overnight in 5% non-fat dry milk in TBST (0.15 M NaCl, 20 mM Tris-HCL, 0.05% Tween 20 (Sigma-Aldrich), pH 7.4) overnight at 4° C. and probed with 5 μg of digoxigenin-labeled C3 or C3b (Advanced Research Technologies, San Diego, Calif., USA). Binding was visualized by incubating with a 1:15,000 dilution of anti-digoxigenin-alkaline phosphatase (AP) conjugated Fab fragments (Roche Diagnostics,: Mannheim, Germany) and developed with 10 ml of 1-Step™ NBT/BCIP solution (Pierce). All incubations were performed in 15 ml of 1% TBST for 1 h with shaking at room temperature and membranes were washed 3 times (5 min with shaking) in TBST between all steps. C3 and C3b were digoxigenin-labeled as described previously according to manufacturer's instructions (36).

Further characterization of the ˜19 kDa C3-binding protein was performed by fractionating 50 μl of supernatant from an overnight SA culture on a large (20 cm) 15% SDS-PAGE gel and transferring onto a PVDF membrane as described above. The membrane was subsequently stained with 0.05% Coomassie brilliant blue R250 solution for 20 min and destained with 50% methanol. Two candidate bands of interest were noted on the membrane and sent to the Protein Chemistry Laboratory (Texas A&M University, College Station, Tex., USA) for N-terminal sequencing which resulted in the identification of residues S-E-G-Y—P—R-E-K-K and F-T-F-E-P-F-P-T-N-E corresponding to the Efb (PubMed accession number Q08691) and the SA1755 (PubMed accession number E89983) protein sequences from SA, respectively.

Cloning of the sac3 and sa a755 genes from S. aureus strain Newman. The sac3, and the sa1755 genes excluding the 5′ signal sequence were amplified by polymerase chain reaction (PCR) using S. aureus strain Newman DNA as a template. The following oligonucleotide primers were used: 5′-CGC GGA TCC CCA AGA GAA AAG AAA CCA GTG AGT A-3′ forward primer and 5′-AAC TGC AGA GTT TTA TTT AAC TAA TCC TTG-3′ reverse primer and 5′-CGC GGA TCC CCG TTT CCT ACA AAT GAA GAA-3′ forward primer and 5′-AAC TGC AGC TAG TAT GCA TAT TCA TTA-3′ reverse primer for sac3 and sa1755, respectively (IDT Inc, Coralville, Iowa, USA). Bam HI and Pst I restriction enzyme sites (underlined) were incorporated into the forward and reverse primers, respectively. Each reaction contained 500 ng of template DNA, 5 pmol of forward and reverse primers, 25 mM dNTPs, 1.5 mM MgCl₂, 16 mM (NH₄)₂SO₄, 67 mM Tris-HCl pH 8.8, 0.01% Tween-20, and 2 units of Taq DNA polymerase (CLP, San Diego, Calif., USA). The reaction was performed on a Perkin-Elmer DNA Thermocycler using the following conditions: 94° C. for 1 min, 52° C. for 1. min, and 72° C. for 1 min for 30 cycles. The resulting PCR amplifications resulted in 400 or 351 base pair products that were subsequently TA-cloned into the pCRT7/NT-TOPO expression vector (Invitrogen, Carlsbad, Calif., USA) and designated pCRT7/NT-SAC3 or pCRT7/NT-SA1755, respectively. Nucleotide sequencing was performed with the Sequenase version 2.0 sequencing kit (US Biochemicals) according to the manufacturer's instructions and by automated sequencing (Molecular Genetics Core Facility in the Department of Microbiology and Molecular Genetics, University of Texas-Houston Medical School). Sequencing of sac3 and sa1755 was performed using the oligonucleotide primers T7 forward 5′-TAA TAC GAC TCA CTA TAG GG-3′ and T7 reverse 5′-CTA GTT ATT GCT CAG CGG TGG -3′ (IDT Inc).

Expression and Purification of Recombinant Proteins. The SA proteins SAC3, SA1755, Map19 (18) and the Staphylococcus epidermidis Fgn-binding protein (SdrG) (29) were expressed as recombinant N-terminal His-tagged proteins that allowed for purification using metal ion-chelating chromatography as described previously (18, 36). Map19 and SdrG were expressed using the pQE expression vector (QIAGEN Inc., Chatsworth, Calif., USA) in E. coil (JM101) (Stratagene, La Jolla, Calif., USA) and SAC3 and SA1755 were expressed using the pCRT7/NT-TOPO (Invitrogen) expression vector in E. coil (BL21) harboring the corresponding plasmids, respectively. Map19 is a secreted SA protein that can be detected in SA supernatants (18) and was used as negative control protein for various assays.

E. coil were grown at 37° C. in Lennox broth (LB) containing the appropriate antibiotics until they reached an A₆₀₀ of 0.6 (37). Isopropyl-□-D-thiogalactopyranoside (IPTG) (Life Technologies) was added to a final concentration of 0.2 mM, and the cells were incubated at 37° C. for an additional 4 h. Cells from a 1 L culture were harvested by centrifugation and resuspended in 10 ml “binding buffer” (BB) (20 mM Tris HCl, 0.5 M NaCl, 15 mM imidazole, pH 8.0) and lysed in a French pressure cell at 11,000 pounds/inch² (36). The lysate was centrifuged at 40,000×g for 15 min and the supernatant filtered through a 0.45 μm filter. A 1 ml iminodiacetic acid Sepharose column (Sigma, St. Louis, Mo.) was charged with 75 mM NiCl₂.H₂O and equilibrated with BB. The filtered supernatant was applied to the column and washed with 10 volumes of BB, then 10 volumes of BB containing 60 mM imidazole. The bound proteins were eluted with BB containing 200 mM imidazole, dialyzed against PBS containing 10 mM EDTA, then dialyzed against PBS (36). Protein concentrations were determined by BCA (Pierce) and proteins were stored at −20° C. until use.

Western-Ligand Blot Analysis. Recombinant proteins (4 μg each), C3b or C3 (2 μg), human Fgn (2 μg) (29), or human serum ( 20 μl of a 1:20 dilution in PBS) (Diamedix, Miami, Fla., USA) were subjected to SDS-PAGE under reducing conditions and visualized with Coomassie brilliant blue or transferred onto a supporting membrane and blocked overnight as described above. All incubations and washes were performed as described above.

Recombinant proteins were probed with either 1 μg or 7.5 μg of digoxigenin-labeled C3b, C3 or Fgn, respectively, prior to incubation with anti-digoxigenin-AP-labeled Fab fragments. Blots were developed as described above.

C3b, Fgn, or human serum were probed with either 80 μg SAC3 (or control proteins) or a 1:2000 dilution of biotin-labeled polyclonal chicken anti-human C3 antibodies. The secondary incubation for recombinant protein-probed blots consisted of a 1:5000 dilution of a monoclonal mouse (IgG2a) anti His antibodies (Amersham Pharmacia Biotech Inc., Piscataway, N.J., USA) followed by a goat anti-mouse AP-labeled secondary antibody (ICN/Cappel Biomedicals Inc., Aurora, Ohio, USA). The secondary incubation for chicken anti-C3-probed blots consisted of a 1:10,000 dilution of avidin-AP (ICN/Cappel). Blots were developed as described above.

Direct Binding ELISA. Immulon-1-microtiter plate wells (Dynatech Laboratories, Chantilly, Va., USA) were coated with either 0.25 μg C3b or Fgn in 50 μl of PBS for 2 h. The plates were washed and then blocked with 200 μl of Super Block (Pierce) for 1 h. After washing, recombinant proteins (0-20 μM in 100 μl final volume/well) were added to corresponding wells and incubated for 1. h. After washing, 100 μl of a 1.5000 dilution of monoclonal mouse anti-His antibodies (Amersham Pharmacia Biotech, Inc.) were added to the wells and incubated for 1 h. After washing, 100 μl of a 1:5000 dilution of goat anti-mouse AP-conjugated antibodies (ICN/Cappel) was added to corresponding wells and incubated for 1 h. After washing, 100 μl of a 1 mg/ml Sigma 104 phosphatase substrate (Sigma) dissolved in 1 M diethanolamine, 0.5 mM MgCl₂, pH 9.8 was added and allowed to develop for 1 h. Plates were read at 405. nm using a microplate reader (Molecular Devices, Menlo Park, Calif., USA). Plates were washed 5 times between all steps with PBS-0.05% Tween 20 and all incubations took place at 37° C. All dilutions were made using Super Block unless otherwise specified.

Complement Activity Assays. The effect of SAC3 on both the classical and alternative pathways was examined. Total serum complement assays or CH50 is the traditional method for determining functional complement activity. A clinical diagnostic kit, EZ Complement CH50 assay kit, (Diamedix) was used to evaluate the effects of recombinant SAC3 on classical complement pathway activation. A 20 μM (20 μl) concentration of either SAC3, or SdrG were incubated with 20 μl of the standard reference serum (Diamedix) at 37° C. for 1 h. 20 μl of each of the standard, high and low reference serum samples were incubated with 20 μl of PBS and incubated in the same fashion as test serum samples. During this incubation, tubes containing a standardized suspension of sheep erythrocytes (sRBC) coated with anti-erythrocyte antibodies were allowed to equilibrate to room temperature. After this incubation, 10 μl of the serum/protein mix or control serum samples were added to individual sRBC-containing tubes and incubated at room temperature for 1 h. The tubes were centrifuged at 1800 rpm for 10 min and the absorbance of the supernatants was read at 405 nm to determine the percent of erythrocyte cell lysis. The data are expressed as percent lysis of the standard reference serum.

Alternative pathway activation was measured using a procedure adapted from Advance Research Technologies. Briefly, the recombinant proteins SAC3 or SdrG (10 μl each at a concentration of 20 μM) were incubated with 10 μl of standard reference serum (Diamedix) at 37° C. for 1 h. To each recombinant protein/serum mixture, 10 μl of 100 mM MgCl₂/EGTA, 10 μl of GVB (gelatin-veronal buffer) (Advanced Research Technologies) (38), and 30 μl of a 5×10⁸/ml rabbit erythrocytes (Advanced Research Technologies) stock were added and incubated at 37° C. for 30 min. Reactions were stopped by adding 1 ml of ice-cold GVB. The tubes were centrifuged at 1800 rpm for 10 min and the absorbance of the supernatants was read at 405 nm to determine the percent of erythrocyte cell lysis. The data are expressed as percent lysis of the complement standard reference serum.

Results

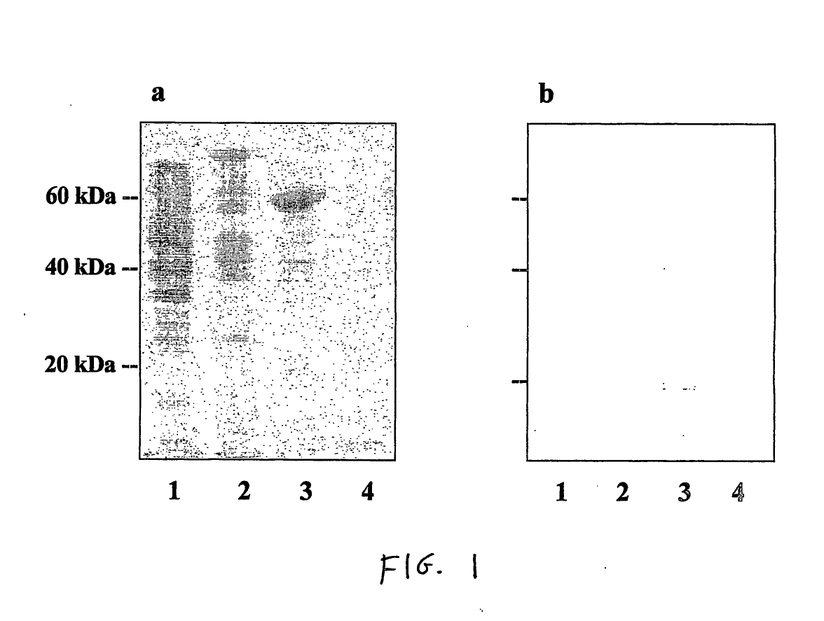

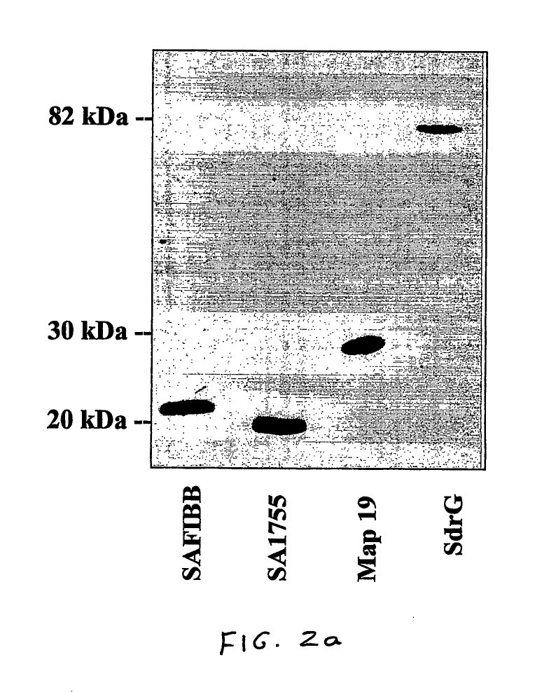

S. aureus Secretes A C3-Binding Protein. To determine if SA expressed any C3-binding proteins, whole cell lysates from E. coli, S. carnosus, S. aureus or S. aureus-supernatants were subjected to SDS-PAGE and stained with Coomassie brilliant blue (FIG. 1a) or transferred onto a PVDF membrane and probed with digoxigenin-labeled C3b (FIG. 1b). An approximately 19 kDa band specific for C3b was detected in both SA whole cell lysates and SA supernatants but not in control cell lysates (FIG. 1b). C3b, which contains a hydrolyzed thioester bond, and not C3 was used as a probe to demonstrate that binding to bacterial proteins was independent of the thioester conformation. However, blots probed with digoxigenin-labeled C3 revealed similar staining and blots probed with an AP-labeled secondary antibody alone revealed no nonspecific binding (data not shown). All subsequent assays were performed with C3b or digoxigenin-labeled C3b as a precautionary measure against nonspecific binding.

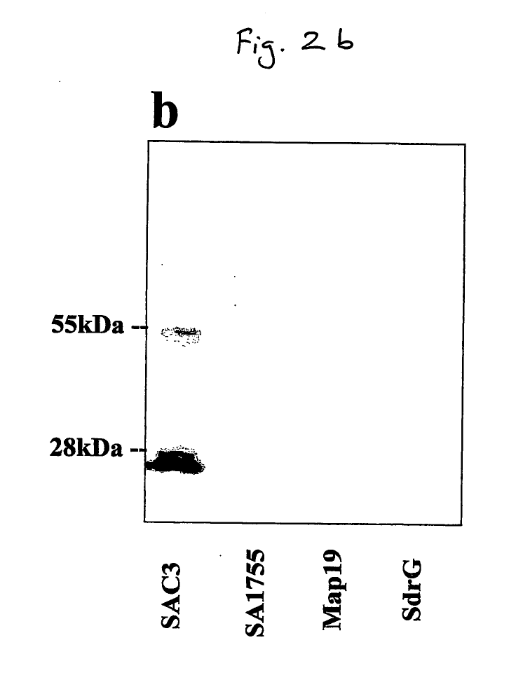

Efb (SAC3) Is The S. aureus C3-Binding Protein. Two candidate proteins with an approximate size of 19 kDa as depicted in FIG. 1b were selected for N-terminal sequencing from Coomassie-stained PVDF membranes. These proteins were identified as the ˜19 kDa and ˜17 kDa Efb (33) and SA1755 proteins, respectively. Efb will be referred to in this report as SAC3. Recombinant His-tagged forms of SAC3 and SA1755 were cloned and expressed (FIG. 2a) and the capacity of these proteins to bind C3b and Fgn was assessed by Western-ligand blot analysis (FIG. 2b-c). Recombinant forms of the secreted SA protein Map19 and the S. epidermidis Fgn-binding protein (SdrG) were used as controls (18, 29). Only SAC3 bound digoxigenin-labeled C3b and both SAC3 and SdrG, but neither SA1755 nor Map19, bound to digoxigenin-labeled Fgn (FIGS. 2b and 2c, respectively).

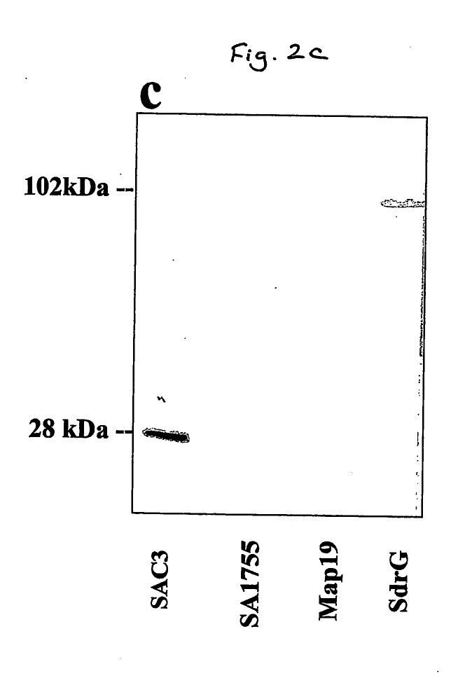

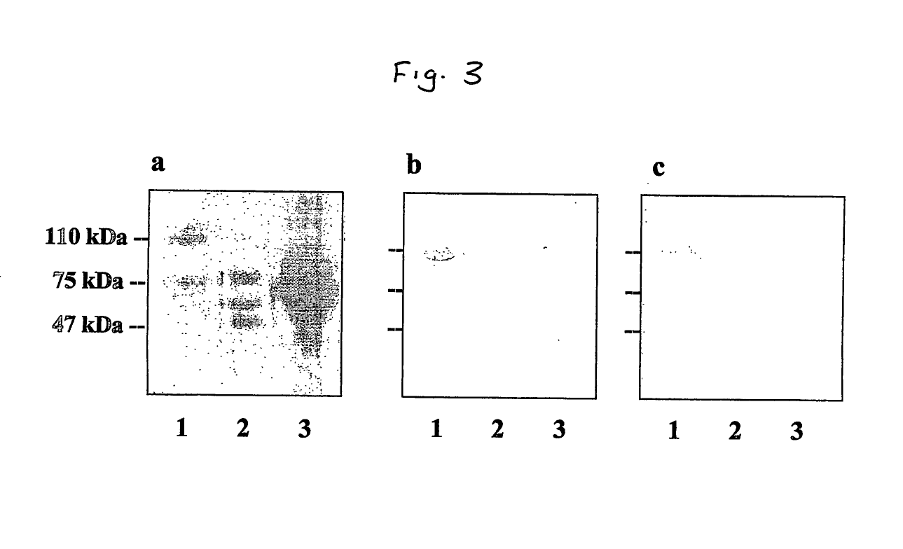

When C3b, Fgn, and human serum were subjected to SDS-PAGE (FIG. 3a, lanes 1-3, respectively), transferred onto to PVDF membranes and probed with either recombinant SAC3 or with anti-human C3 antibodies (FIGS. 3b and 3c, respectively) distinct binding to the C3 □chain was observed (FIGS. 3b and 3c, lanes 1 and 3). The minor shift in molecular weight observed between the C3b □-chain and the C3 □-chain detected in human serum (FIGS. 3b and 3c, lanes 1 and 3, respectively) is due to the proteolytic cleavage of the 77-residue peptide (C3a) removed from the amino terminus of the C3 □-chain that results in the formation of C3b.

Confirmation that SAC3 was binding specifically to C3 in the human serum sample was obtained by probing a corresponding blot with anti-human C3 antibodies (FIG. 3c). Blots probed with either SAC3 or anti-human C3 antibodies revealed binding to bands of identical molecular weights, suggesting that in human serum SAC3 only, bound C3. Blots probed with SA1755 or with secondary antibody alone revealed no cross-reactive binding (data not shown). SAC3-Fgn binding was less conspicuous and only very weak binding to the □ and □ chains was detected (FIG. 3b, lane 2). The weak Fgn binding by SAC3 is somewhat contradictory to previous reports that suggested a strong binding to the Fgn □-chain (34, 39).

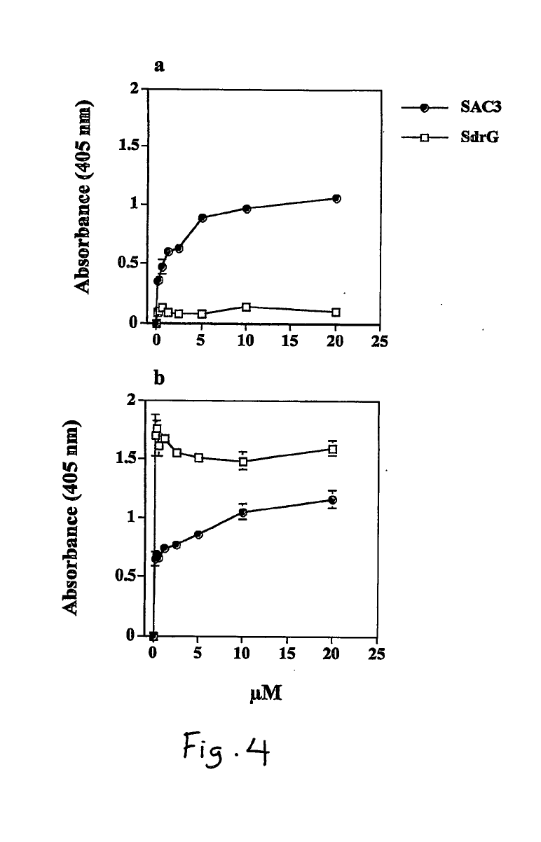

Further binding analysis revealed a dose-dependent and saturable binding of SAC3 to both C3b- or Fgn-coated microtiter wells compared to SdrG that bound only to Fgn-coated wells (FIG. 4a-b). Neither SdrG (FIG. 4a) nor-SA1755 (data not shown) bound to C3b-coated wells. One possibility for the differences in SAC3 binding to Fgn between Western-ligand blots and by ELISA may be due to a different conformational status of the Fgn in the two assays. In the Western-ligand blots, the Fgn may be at least partly denatured (following SDS-PAGE) when the bacterial protein encounters Fgn, whereas a properly folded form of Fgn may be the target of SAC3 binding in the ELISA-type assay.

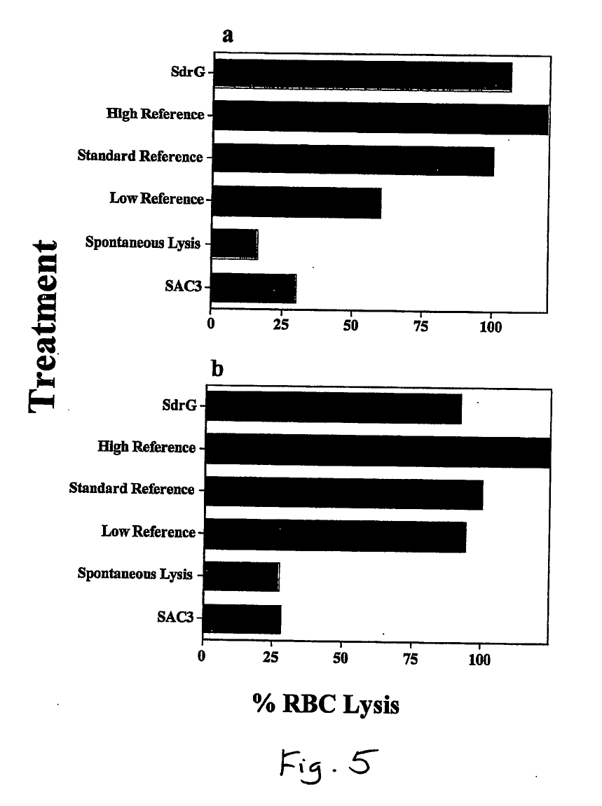

SAC3 Interferes With Alternative And Classical Pathways. Although direct binding between SAC3 and C3 had been established, it remained unclear what function if any SAC3 served with regard to complement activation. Because C3 is a key initiating protein for the alternative, classical and MBL complement pathways, C3 inactivation would essentially eliminate all complement activity and provide a potent immunomodulatory component for SA. Functional assays for both the classical and alternative pathways were used to assess the capacity of SAC3 to interfere with complement activity. Complement-mediated lysis of red blood cells by either the classical or alternative pathways was inhibited if human serum was preincubated with SAC3 but not with control proteins (FIG. 5a and 5b, respectively), suggesting that SAC3 can function as a complement inhibitory protein by binding to C3. SA1755 had no complement function inhibitory properties (data not shown).

Discussion

The broad spectrum of diseases which can result from SA infections is a function of various parameters: the bacterium's versatility allowing for the colonization of various tissues and avoidance of host immunity, the genetic make up and immune status of the host, and the portal of entry (e.g. intravenous, cutaneous, or intraperitoneal) (1, 18, 40, 41). Complement is an essential component of the innate immune system and in higher vertebrates and mammals increased bacterial and viral infections are concomitant with complement protein deficiencies (23). It is therefore not surprising that numerous human pathogens from diverse genera have developed complement-evasion strategies (23-26).

SA employs various immune evasion strategies: Protein A and Map can interfere with antibody and memory T cell responses, respectively, and super antigens (bacterial toxins) can nonspecifically activate up to 20% of naive T cells resulting in the release of large quantities of cytokines and generating a clinical condition resembling septic shock (1, 18).

This work describes another SA MIM (SAC3) that binds to C3 and interferes with complement activation. Although SAC3 had been previously characterized as a Fgn-binding protein (Efb) (31, 32), it is not unusual for bacterial proteins to have multiple biological activities (e.g. SA Map and Streptococcal M protein) (15-18, 21, 22) and may be one explanation for the capacity of these bacteria to colonize, survive and persist in vastly different environments (e.g. skin, blood, kidney, or bone) and cause such a broad range of diseases.

A large number of human pathogens have evolved mechanisms of complement evasion (23, 42, 43). However, the human pathogens that generate specific C3-binding proteins is limited to a relatively small group that includes Trypanosoma cruzi, Streptococcus pneumoniae, Pseudomonas aeruginosa, Mycobacterium tuberculosis, Mycobacterium leprae, Leishmania major, Legionella pneumophila, Chlamydia trachomatis and herpes simplex virus (22, 44-51). What role each of these proteins plays in bacterial survival or evasion of host defenses is not completely understood. M. leprae, L. major, and L. pneumophila use C3 as a ligand to facilitate their internalization into mononuclear cells via CR1 and CR3 complement receptors (44, 49, 50). What role the C3-binding proteins from C. trachomatis or M. tuberculosis play has not been defined clearly, although published reports do not suggest an immunomodulatory function for these proteins (47, 52). T. cruzi, S. pneumoniae, P. aeruginosa and herpes simplex virus type 1 and type 2 can all generate C3-binding proteins with complement inhibitory properties (45, 48, 53-56). It is suggested that the surface glycoprotein C from herpes, simplex viruses inhibits alternative pathway activation via-blocking the binding of properdin to° C3b of the C3 convertase (53-55) and the shed T. cruzi CRPs (complement regulatory proteins) can inhibit alternative complement activation by molecular mimicry of the mammalian DAF (decay accelerating factor) (48, 57). P. aeruginosa secretes a C3 protease which can inactivate C3 by cleaving the □-chain (51), and the CbpA protein (choline-binding protein) from S. pneumoniae likely plays an anti-opsonization/phagocytosis role by binding to C3 (45).

SAC3 represents the first complement regulatory MIM identified from SA and in conjunction with other SA immune evasion mechanisms may further explain the capacity of SA to cause persistent infections. It is interesting that SA and P aeruginosa are the only bacteria capable of generating alternative and classical pathway-inhibitor proteins since both are opportunistic pathogens capable of causing many different disease manifestations.

Although the mechanism by which SAC3 inhibits complement activity is not yet known, it is likely to function by sterically hindering the activation of C3 by preventing its binding to activator surfaces (alternative pathway) or C3 convertase (classical pathway) since C3 incubated in the presence of SAC3 for 1 h at 37° C. did not result in any discernable degradation products following examination by SDS-PAGE (data not shown). Previous work demonstrated that SA capsular proteins and cell-wall peptidoglycans can provide activator surfaces that can activate the alternative pathway (58, 59), suggesting that complement regulatory proteins like SAC3 may play an important role in SA survival by diminishing or preventing complement activation. That SAC3 is highly conserved among and expressed on all SA strains examined suggests that this protein is critical to SA survival (60). To our knowledge, SAC3 is the first SA MIM with complement inhibitory properties and therefore represents a novel member of SA's growing immunomodulatory arsenal.

### REFERENCES

The following articles referred to above are incorporated herein by reference as if set forth herein their entirety:
1. Lowy, F. D. (1998) N. Engl. J. Med. 339, 520-532.
2. Kissane, J. M. (1997) in Pathology of Infectious Diseases, eds. Connor, D. H., Chandler, F. W., Schwartz, H. J., Manz, H. J. & Lack, E. E. (Appleton and Lange, Samford, Conn.), Vol. I, pp. 805-816.
3. Hiramatsu, K., Cui, L., Kuroda, M. & Ito, T. (2001) Trends Microbiol 9, 486-93.
4. Boden, M. K. & Flock, J. I. (1989) Infect Immun 57, 2358-63.
5. Chhatwal, G. S., Preissner, K. T., Muller-Berghaus, G. & Blobel, H. (1987) Infect Immun 55, 1878-83.
6. Foster, T. J. & Höök, M. (1998) Trends Microbiol 6, 484-8.
7. Herrmann, M., Suchard, S. J., Boxer, L. A., Waldvogel, F. A. & Lew, P. D. (1991) Infect Immun 59, 279-88.
8. Hudson, M. C., Ramp, W. K. & Frankenburg, K. P. (1999) FEMS Microbiol Lett 173, 279-84.
9. Liang, O. D., Ascencio, F., Vazquez-Juarez, R. & Wadstrom, T. (1993) Zentralbl Bakteriol 279, 180-90.
10. Lopes, J. D., dos Reis, M. & Brentani, R. R. (1985) Science 229, 275-7.
11. Moreillon, P., Entenza, J. M., Francioli, P., McDevitt, D., Foster, T. J., Francois, P. & Vaudaux, P. (1995) Infect Immun 63, 473843.
12. Sinha, B., Francois, P. P., Nusse, O., Foti, M., Hartford, O. M., Vaudaux, P., Foster, T. J., Lew, D. P., Herrmann, M. & Drause, K. H. (1999) Cell Microbiol 1, 101-117.
13. Vercellotti, G. M., McCarthy, J. B., Lindholm, P., Peterson, P. K., Jacob, H. S. & Furcht, L. T. (1985) Am J Pathol 120, 13-21.
14. Wann, E. R., Gurusiddappa, S. & Höök, M. (2000) J Biol Chem 275, 13863-71.
15. Chavakis, T., Hussain, M., Kanse, S. M., Peters, G., Bretzel, R. G., Flock, J. I., Herrmann, M. & Preissner, K. T. (2002) Nat Med 8, 687-93.
16. Jahreis, A., Yousif, Y., Rump, J. A., Drager, R., Vogt, A., Peter, H. H., & Schlesier, M. (1995) Clin Exp Immunol 100, 406-11.
17. Jahreis, A., Beckheinrich, P. & Haustein, U. F. (2000) Br J Dermatol 142, 680-7.
18. Lee, L. Y., Miyamoto, Y. J., McIntyre, B. W., Höök, M., McCrea, K. W., McDevitt, D. & Brown, E. L. (2002) J Clin Invest 110, 1461-1471.
19. Renno, T., Attinger, A., Locatelli, S., Bakker, T., Vacheron, S. & MacDonald, H. R. (1999) J Immunol 162, 6312-5.
20. Kreikemeyer, B., McDevitt, D. & Podbielski, A. (2002) Int J. Med. Microbiol. 292, 283-295.
21. Jonsson, K., McDevitt, D., McGavin, M. H., Patti, J. M. & Hook, M. (1995) J Biol Chem 270, 21457-60.
22. Horstmann, R. D., Sievertsen, H. J., Leippe, M. & Fischetti, V. A. (1992) Infect Immun 60, 5036-41.
23. Prodinger, W. M., Wurzner, R., Erdei, A. & Dierich, M. P. (1999) in Fundamental Immunology, ed. Paul, W. E. (Lippincott-Raven, Philadelphia), pp. 967-995.
24. Wurzner, R. (1999) Mol Immunol 36, 249-60.
25. Lachmann, P. J. (2002) Proc Natl Acad Sci USA 99, 8461-2.
26. Hornef, M. W., Wick, M. J., Rhen, M. & Normark, S. (2002) Nat Immunol 3, 1033-40.
27. Haviland, D. L. & Wetsel, R. A. (1999) in Encyclopedia of Molecular Biology, ed. Creighton, T. E. (John Wiley & Sons, Inc., New York), pp. 525-532.
28. Sakiniene, E., Bremell, T. & Tarkowski, A. (1999) Clin Exp Immunol 115, 95-102.
29. Davis, S. L., Gurusiddappa, S., McCrea, K. W., Perkins, S. & Höök, M. (2001) J Biol chem 276, 27799-27805.
30. Ni Eidhin, D., Perkins, S., Francois, P., Vaudaux, P., Höök, M. & Foster, T. J. (1998) Mol Microbiol 30, 245-57.
31. Bodén, M. K. & Flock, J. I. (1989) Infect Immun 57, 2358-63.
32. Bodén, M. K. & Flock, J. I. (1992) Microb Pathog 12, 289-98.
33. Bodén, M. K. & Flock, J. I. (1994) Mol Microbiol 12, 599-606.
34. Palma, M., Shannon, O., Quezada, H. C., Berg, A. & Flock, J. I. (2001) J Biol Chem 276, 31691-7.
35. Brown, E. L., Guo, B. P., O'Neal, P. & Höök, M. (1999) J Biol. Chem 274, 26272-8.
36. Guo, B. P., Brown, E. L., Dorward, D. W., Rosenberg, L. C. & Höök, M. (1998) Mol Microbiol 30, 711-23.
37. Maniatis, T., Fritsch, E. F. & Sambrook, J. (1989) Molecular Cloning: A Laboratory Manual (Cold Spring Harbor Laboratory, Cold Spring Harbor, N.Y.).
38. Collard, C. D., Montalto, M. C., Reenstra, W. R., Buras, J. A. & Stahl, G. L. (2001) Am J Pathol 159, 1045-54.
39. Palma, M., Wade, D., Flock, M. & Flock, J. I. (1998) J Biol Chem 273, 13177-81.
40. Tacconelli, E., Tumbarello, M. & Cauda, R. (1998) N Engl J Med 339, 2026-7.
41. Lina, G., Piemont, Y., Godail-Gamot, F., Bes, M., Peter, M. O., Gauduchon, V., Vandenesch, F. & Etienne, J. (1999) Clin Infect Dis 29, 1128-32.
42. Joiner,: K. A. (1988) Annu Rev Microbiol 42, 201-30.
43. Cooper, N. R., (1991) Immunol Today 12, 327-31.
44. Bellinger-Kawahara, C. & Horwitz, M. A. (1990) J Exp Med 172, 1201-10.
45: Cheng, Q., Finkel, D. & Hostetter, M. K. (2000) Biochemistry 39, 5450-7.
46. Lubinski, J. M., Wang, L., Soulika, A. M., Burger, R., Wetsel, R. A., Colten, H., Cohen, G. H., Eisenberg, R. J, Lambris, J. D. & Friedman, H. M. (1998) J Virol 72, 8257-63.
47. Muller-Ortiz, S. L., Wanger, A. R. & Norris, S. J. (2001) Infect Immun 69, 7510-7511.
48. Norris, K. A. (1996) Microb Pathog 21, 23548.
49. Puentes, S. M., Sacks, D. L., da Silva, R. P. & Joiner,. K. A. (1988) J Exp Med 167, 887-902.
50. Schlesinger, L. S. & Horwitz, M. A. (1990) J Clin Invest 85,1304-14.
51. Hong, Y. Q. & Ghebrehiwet, B. (1992) Clin Immunol Immunopathol 62, 133-8.
52. Hall, R. T., Strugnell, T., Wu, X., Devine, D. V. & Stiver, H. G. (1993) Infect Immun61, 1829-34.
53. Friedman, H. M., Wang, L., Pangburn, M. K., Lambris, J. D. & Lubinski, J. (2000) J Immunol 165, 4528-36.
54. Fries, L. F., Friedman, H. M., Cohen, G. H., Eisenberg, R. J., Hammer, C. H. & Frank, M. M. (1986) J Immunol 137, 1636-41.
55. Lubinski, J. M., Jiang, M., Hook, L., Chang, Y., Sarver, C., Mastellos, D., Lambris, J. D., Cohen, G. H., Eisenberg, R. J. & Friedman, H. M. (2002) J Virol 76, 9232-41.
56. Norris, K. A., Bradt, B., Cooper, N. R. & So, M. (1991) J Immunol 147, 2240-7.
57. Norris, K. A. & Schrimpf, J. E. (1994) Infect Immun 62, 236-43.
58. Verbrugh, H. A., Van Dijk, W. C., Peters, R., Van Der Tol, M. E. & Verhoef, J. (1979) Immunology 37, 615-21.
59. Cunnion, K. M., Lee, J. Q. & Frank, M. M. (2001) Infect Immun 69, 6796-803.
60. Bodén Wastfelt, M. K. & Flock, J. I. (1995) J Clin Microbiol 33, 2347-52.

### Example 2

### Experimental Procedures for Isolating the C3 Binding Region of EFB

Overview

The secreted S. aureus extracellular fibrinogen-binding protein (Efb) is a virulence factor that can bind to the complement component C3 and fibrinogen. We have previously demonstrated that the binding of C3 by Efb inhibited complement activation and blocked opsonophagocytosis. In this study, we have identified and characterized the C3-binding region of Efb. By using truncated recombinant forms of Efb we confirmed that the C3-binding region of Efb is located in the C-terminal region of the protein in contrast to the fibrinogen-binding region which is located in the N-terminal region. Furthermore, we have identified the minimal Efb-binding region in C3 to the C3d fragment. Because Efb can bind to both C3,and fibrinogen, the possibility that binding to both ligands occurred simultaneously was examined. Data presented in this report suggest that Efb can bind to C3 and fibrinogen at that same time to form a tri-molecular complex.

Introduction

Staphylococcus aureus is a major health threat in both hospital and community settings (1,2). S. aureus is a commensal organism of the skin and anterior nares, but trauma to the skin barrier can allow for access to host cell tissues where colonization and infection can occur. The result of infection can vary from minor skin infections to lethal systemic diseases such as sepsis, pneumonia, or endocarditis (3). One explanation for the ubiquitous existence of this pathogen, be it in a commensal or infectious state, is the ability of S. aureus to manipulate or evade host immune defense mechanisms. The production of microbial immunomodulatory molecules (MIMs) e.g. protein A, the enterotoxins, and the MHC class II analog protein (Map), provide S. aureus with an arsenal of defense mechanisms against host adaptive immune responses (4-7). Although the effector molecules which drive S. aureus evasion of host “adaptive” immune defenses are better defined, the evasion of host “innate” immunity is less well established.

The complement system plays a central role in innate immunity. Activation of the complement pathways (i.e. Classical, Mannose-binding lectin or Alternative pathways) lead to opsonization and phagocytosis of invading pathogens and, in a majority of cases, directs their lysis and cell death (8). The complement protein C3 is the most critical component in all three complement pathways (9-11). C3 is not only essential in host defense to infection, but it is also plays a vital role in the interface between, innate and acquired immune responses (8). Patients with a C3 deficiencies have a significantly impairment complement system and these individuals have an increased susceptibility to infection (12-14). The direct or indirect targeting of C3 as a mechanism to evade and inhibit complement activation has been used by other pathogens such as Trypanosoma cruzi and Streptococcus pyogenes (15,16). Research on, the interactions between S. aureus and complement activation have mainly revolved around the protective effect that capsule formation affords S. aureus by preventing C3 deposition and C3-mediated opsonization (17-19). As was demonstrated above in Example 1, we recently demonstrated that S. aureus can also ‘actively’ affect complement activity by secreting a C3-binding protein (Efb). Efb is a secreted, constitutively expressed protein that is found at a high incidence among clinical isolates of S. aureus (21,22). The immunomodulatory properties of Efb provided evidence that S. aureus can directly affect innate host defenses. Efb interferes with innate immunity by inhibiting both the classical and alternative complement pathways via a mechanism that involves Efb binding to the complement protein C3 and blocking C3 from binding to activator surfaces, therefore preventing C3 deposition and opsonization (20). In addition, we demonstrated that Efb inhibited complement-mediated opsonophagocytosis.

The N-terminal region of Efb has been described as having fibrinogen-binding activity, consists of two 22-amino acid repeats that have homology to the C-terminus fibrinogen-binding regions of coagulase (21). It is also hypothesized that Efb contains a second fibrinogen-binding region that resides in the C-terminal end of the protein, however, the fibrinogen-binding activity of this ‘region’ are less well defined because binding was dependant on the nature in which fibrinogen is presented (i.e. ???) (23,24). The physiological roles of Efb have been examined in a rat wound healing model and in assays measuring platelet activation, respectively (25-27). Rats infected with wild-type strain S. aureus presented with a higher severity of disease and a reduced wound-healing capacity compared to the Efb mutant strain of S. aureus. The effects of Efb on platelet function are less-well defined, however, it was hypothesized that Efb inhibited platelet aggregation by via its interaction with fibrinogen and the platelet receptor GPIIb/IIIa (26,27).

Experimental Procedures

Cloning of Efb Truncations from S. aureus strain Newman—The efb gene and efb truncations were amplified by polymerase chain reaction (PCR) using S. aureus strain Newman DNA as a template. The following oligonucleotide primers were used: 5′-CGC GGA TCC CCA AGA GAA AAG AAA CCA GTG AGT A-3′ forward primer and 5′-AAC TGC AGA GTT TTA TTT AAC TAA TCC TTG-3′ reverse primer, 5′-CGC GGA TCC CCA AGA GAA AAG AAA CCA GTG AGT A-3′ forward primer and 5′-AAC TGC AGT TAT TCT CTC ACA AGA TTT TGA GCT TG-3′ reverse primer, and 5′-CCA GCA GCG AAA ACT GAT GCA ACT-3′ forward primer and 5′-AAC TGC AGA GTT TTA TTT AAC TAA TCC TTG-3′ reverse primer for rEfb, rEfb 120 and rEfb 165, respectively (IDT Inc., Coralville, Iowa, USA). The resulting PCR products were subsequently cloned using the TA Expression Kit into the pCRT7/NT-TOPO expression vector (Invitrogen, Carlsbad, Calif., USA) and designated pCRT7/NT-rEfb, pCRT7/NT-rEfb120, and pCRT7/NT-rEfb165. Nucleotide sequencing of rEfb, rEfb120 and rEfb165 were performed by automated sequencing (Molecular Genetics Core Facility, University of Texas-Houston Medical School).

Expression and Purification of Recombinant Proteins—The recombinant proteins rEfb, rEfb120, and rEfb165 were expressed as recombinant N-terminal His-tagged proteins that allowed for purification using metal ion-chelating chromatography as described previously (28,29). rEfb, rEfb120, and rEfb165 were expressed using the pCRT7/NT-TOPO (Invitrogen) expression vector in. E. coli (BL21) harboring the corresponding plasmids, respectively. Proteins were expressed and purified as previously described (28,30). Protein concentrations were determined by UV spectroscopy and proteins were stored at −20° C. until use.

Western Blot Analysis—Recombinant proteins (4 μg each), C3b, iC3b, C3c, and C3d (Advanced Research Technologies, San Diego, Calif.) (4 μg) were subjected to SDS-PAGE and examined by staining with 0.05% Coomassie brilliant blue or electro-transferred onto a 0.45 μm Immobilon-P™ PVDF (polyvinylidene fluoride) membrane (Millipore, Bedford, Mass., USA) as described previously (31).

Membranes subjected to Western blot analysis were blocked overnight at 4° C. in 5% non fat dry milk in TBST (0.15 M NaCl, 20 mM Tris-HCL, 0.05% Tween 20 (Sigma-Aldrich, St. Louis, Mo., USA), pH 7.4) and probed accordingly and then developed with 10 ml of 1-Step™ NBT/BCIP solution (Pierce Chemical Co., Rockford, Ill., USA). All incubations were performed in 15 ml of 1% TBST for 1 h with shaking at room temperature and membranes were washed in TBST between all steps. Labeling with digoxigenin was performed as described previously according to the manufacturer's instructions (31).

ELISA-Type Binding Assays—Immulon-1B microtiter plate wells (Dynatech Laboratories, Chantilly, Va., USA) were coated with either 0.25 μg C3b or fibrinogen in 100 μl of PBS overnight at 4° C. The plates were washed and blocked with 200 μl of Super Block (Pierce) for 1 h. Recombinant proteins (0-500 nM in 100 μl final volume/well) were added to the wells and incubated for 1 h. In the next step, 100 μl of anti-His antibodies, (Amersham, ???) (1:5000) were added and incubated for 1 h, followed by 100 μl of goat anti-mouse alkaline phosphatase (AP)-conjugated antibodies (1:5000). Alternatively for digoxigenin-labeled proteins, 100 μl of AP-conjugated anti-digoxigenin antibodies ([Fab fragment] Roche Diagnostics, Mannheim, Germany) (1:5000) were added. Next, 100 μl of a 1 mg/ml Sigma 104 phosphatase substrate (Sigma) dissolved in 1 M diethanolamine, 0.5 mM MgCl₂, pH 9.8 was added and the plates were allowed to develop for 1 h. Plates were read at 405 nm using a microplate reader (Molecular Devices, Menlo Park, Calif., USA). Plates were washed between all steps with PBS-0.05% Tween 20 and all incubations took place at 37° C. All dilutions were made using Super Block unless otherwise specified.

Complement Activity Assays—The EZ Complement CH50 clinical diagnostic assay kit, (Diamedix, Miami, Fla., USA) was used to evaluate the effects of the rEfb truncations on classical complement pathway activation and used as described by the manufacturer. Briefly, human serum (5 μl of complement-reference serum) was incubated in the presence of 5 μg of each recombinant protein at a final volume of 20 μl at 37° C. for 1 h prior to a 1 h incubation at room temperature with antibody-coated sheep RBCs (red blood cells) (3 ml). After incubating, the RBCs were centrifuged (1800 rpm for 10 min) and the absorbance of the supernatants (150 μl) was measured at 405 nm using a microplate reader as described above to determine the percent lysis of each sample. The data are expressed as percent lysis of the standard reference serum and the values were derived using the equation: Absorbance of Sample/Absorbance of Reference×CH50 Value of Reference (Diamedix).

Results

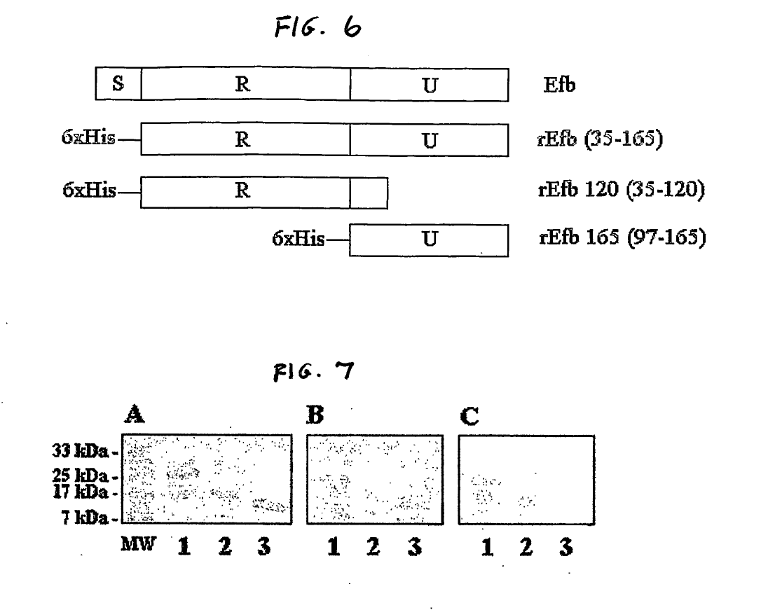

The C-terminal Region of Efb Binds C3b but not to Fibrinogen—To define the general binding regions of Efb with respect to C3- and fibrinogen-binding, respectively, two recombinant, truncated forms of Efb containing the N-terminal region (amino acids [A.A.] 35-120) or C-terminal region (A.A. 97-165) of the protein were generated and designated as rEfb120 and rEfb165, respectively (FIG. 6). C3- and fibrinogen-binding was first assessed by Western-ligand blot analysis. rEfb (full-length), rEfb120 (N-terminal region of Efb), and rEfb165 (C-terminal region of Efb) were subjected to SDS-PAGE and Coomassie-stained (FIG. 7A) or transferred onto a PVDF membrane and probed with digoxigenin-labeled C3b (FIG. 7B) or digoxigenin-labeled fibrinogen (FIG. 7C). Western blots probed with C3b revealed that rEfb and rEfb165 bound C3b compared to rEfb120, which did not bind to C3b (FIG. 7B). Conversely, Western blots probed with fibrinogen demonstrated binding to rEfb and rEfb120, but not to rEfb165 (FIG. 7C). Blots probed with an AP-labeled secondary antibody alone revealed no color change following the addition of substrate (data not shown).

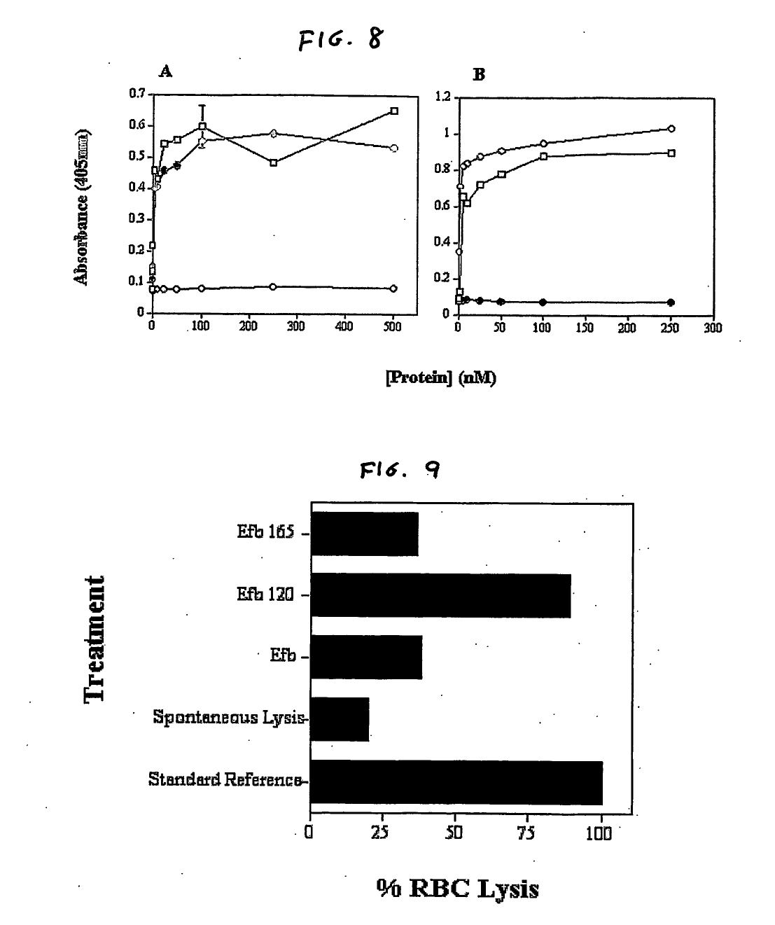

Similar results were obtained using ELISA-type binding assays. rEfb and rEfb165 bound to plate-bound C3b and rEfb and rEfb120 bound plate-bound fibrinogen in a dose-dependent and saturable manner (FIG. 8A-B).

The C-terminal Region of Efb Inhibits Complement Activation—We have previously demonstrated that Efb interferes with in vitro assays testing for *complement activity (20). To determine if the binding between. rEfb165 and C3 could also affect complement activity, we examined the effect of rEfb165 in a functional assay for the classical complement pathway which employs RBC lyses as a read-out for complement activity. Data presented in FIG. 9 demonstrate that both full-length Efb and Efb165 could inhibit complement-mediated lysis of RBCs to the same degree compared to Efb120 which had no inhibitory activity.

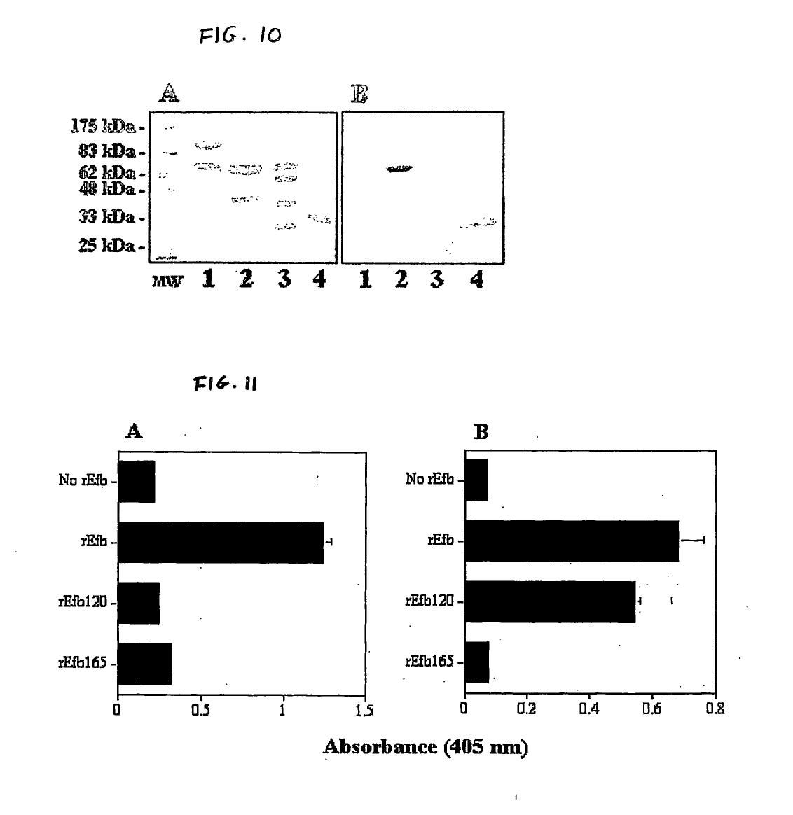

Efb Binds to the C3d Fragment of C3—Additional experiments designed to further examine the interaction(s) between Efb and C3 were performed by mapping the binding site in C3 for Efb. During complement activation process, C3 undergoes multiple cleavage events which generate various C3 fragments which include C3b, iC3b, C3c, C3d, C3dg, and C3g (11). To determine which C3 fragment binds rEfb, the C3 protein fragments C3b, iC3b, C3c, and C3d were subjected to SDS-PAGE and Coomassie-stained (FIG. 10A) or transferred to PVDF and probed with rEfb (FIG. 10B). Western-ligand blot analysis demonstrated that rEfb bound to C3d and all C3d-containing fragments. C3c, which does not contain the C3d region, did not bind Efb (FIG. 10B). Blots probed with secondary antibody or with secondary antibody only revealed no color change following the addition of substrate (data not shown).

rEfb Binds to C3 and Fibrinogen, Simultaneously—Data presented in this report suggest that Efb binds could to C3 and fibrinogen at two distinct sites. Due to this dual-binding capacity of Efb, we investigated the possibility that Efb can bind to both C3 and fibrinogen simultaneously. A sandwich-type ELISA was performed were fibrinogen was immobilized in microtiter wells and probed first with either rEfb, rEfb120, or rEfb165, then subsequently with digoxigenin-labeled C3b (FIG. 11). These results demonstrated that rEfb but not the rEfb truncated proteins rEfb120 and rEfb165 can bind to C3 and fibrinogen simultaneously (FIG. 11A). As a control, anti-His antibodies were used to detect the recombinant proteins that bound to fibrinogen (FIG. 11B). As expected rEfb and rEfb120 bound to plate-bound fibrinogen in contrast to rEfb165 which demonstrated no detectable binding, (FIG. 11B and FIG. 7C).

Discussion

For S. aureus to become a successful pathogen, it must rely upon three main steps during the infection process. S. aureus must first gain access to host cell tissues for adherence and colonization; secondly, avoid host immunity; and third, disseminate to other tissues. The production of immunomodulatory proteins such as Efb assists in evading the innate “arm” of the host immune response by inhibiting the complement pathways. In addition to the C3 binding properties of Efb, it can also bind to fibrinogen and may also function in aiding in bacterial adherence, clot formation or negatively affect wound healing (25-27).

In this study, we have identified and characterized the C3 binding region in Efb. The binding region has been located to the C-terminus of Efb whereas the fibrinogen binding region resides in the N-terminus of the protein. In our investigation, the C-terminal recombinant Efb protein (rEfb165) exclusively bound to C3, which is not in agreement with other studies that have shown Efb to contain a second fibrinogen binding region, within the same region of the protein (24,26). Both ELISA and western blot analysis show that rEfb165 bound specifically to C3 and not to fibrinogen. DNA sequence analysis of the cloned refb165 was consistent with sequence data identified in the NCBI nucleotide database (Q08691) and does not contain any mismatches or mutations. Our discrepancy in the binding affinity for C3. and the lack of binding to fibrinogen by rEfb165 is unknown. Previous studies have relied on peptide analysis and binding of plate bound Efb versus plate bound fibrinogen which maybe a source of disparity (24). Initial studies investigating Efb and fibrinogen binding at the C-terminal region indicated that binding of Efb to fibrinogen occurs only to solid-phase but not soluble fibrinogen (21). This may indicate a structural reliance on fibrinogen in order to bind the C-terminal region of Efb and maybe an explanation for the difference in binding.

Microbial proteins that can bind to extracellular matrix and to the complement proteins are not exclusive to Efb. The M protein produced by Group A Streptococcus is a surface expressed protein that can bind to fibrinogen and the complement regulatory protein factor H (32-35). The binding of fibrinogen and factor H serves to function as an immune evasion mechanism by Streptococcus to inhibit phagocytosis and C3 deposition, respectively (33). We have demonstrated that Efb binds to fibrinogen and C3 at two distinct locations and have explored the potential of Efb to concurrently bind C3 and fibrinogen. By performing a sandwich-type ELISA, we have shown that Efb can simultaneously bind to C3 and fibrinogen. The underlying principle for Efb to bind to both proteins concurrently is unknown, but it may function to negatively affect both complement activation and fibrinogen mediated pathways (coagulation and platelet aggregation) concurrently since the two pathways are interrelated (36).

In addition to identifying the minimal C3-binding region of Efb, we extended our studies to include identifying the minimal binding region located within C3. We have demonstrated that Efb binds specifically to the C3d fragment of C3. The C3d fragment of C3 contains important binding sites for factor H and the complement receptor 2 (CR2) (11). CR2 is expressed on B cells, T cells, and follicular dendritic cells and is implicated in the regulation of B and T cell responses (37). If Efb bound C3d blocks binding to CR2, this may serve as an additional mechanism that can modulate B and T cell immune response through that receptor.

The identification and characterization of the C3 and Efb binding regions have been elucidated in this study. Identifying the C3-binding region of Efb and its ability to inhibit complement activity may potentially be advantageous in developing clinical therapeutics in the treatment of complement-mediated disorders such as systemic lupus erythematosus and autoimmune arthritis (38), as well as hemolytic anemia.

### REFERENCES

The following articles referred to above in Example 2 are incorporated herein by reference as if set forth herein their entirety:

### REFERENCES

1. Hiramatsu, K., Cui, L., Kuroda, M., and Ito, T. (2001) Trends Microbiol.9, 486-493
2. Lowy, F. D. (1998) N Engl J Med 339, 520-532
3. Kissane, J. M. (1997) in Pathology of Infectious Diseases (D. H. Connor, F. W. C., H. J. Schwartz, H. J. Manz and E. E. Lack, ed) Vol. 1, pp. 805-816, Appleton and Lange, Stamford, Conn., USA
4. Peterson, P. K., Verhoef, J., Sabath, L. D., and Quie, P. G. (1977) Infect Immun 15, 760-764
5. Proft, T., and Fraser, J. D. (2003) Clin Exp Immunol 133, 299-306
6. Lee, L. Y., Miyamoto, Y. J., McIntyre, B. W., Hook, M., McCrea, K. W., McDevitt, D., and Brown, E. L. (2002) J Clin Invest 110, 1461-1471
7. Chavakis, T., Hussain, M., Kanse, S. M., Peters, G., Bretzel, R. G., Flock, J. I., Herrmann, M., and Preissner, K. T. (2002) Nat Med 8, 687-693
8. Walport, M. J. (2001) N Engl J Med344, 1058-1066
9. Lambris, J. D. (1988) Immunol Today 9, 387-393
10. Lambris, J. D., and Muller-Eberhard, H. J. (1986) Mol Immuno 23, 1237-1242
11. Sahu, A., and Lambris, J. D. (2001) Immunol Rev 180, 3548
12. Botto, M., and Walport, M. J. (1993) Int Rev Immunol 10, 37-50
13. Peleg, D., Harit-Bustan, H., Katz, Y., Peller, S., Schlesinger, M., and Schonfeld, S. (1992) Pediatr Infect Dis J 11, 401-404
14. Totan, M. (2002) Indian J Pediatr 69, 625-626
15. Norris, K. A., Bradt, B., Cooper, N. R., and So, M. (1991) J Immunol 147, 2240-2247
16. Pandiripally, V., Gregory, E., and Cue, D. (2002) Infect Immun 70,6206-6214
17. Cunnion, K. M., Zhang, H. M., and Frank, M. M; (2003) Infect Immun 71, 656-662
18. Cunnion, K. M., Lee, J. C., and Frank, M. M. (2001) Infect Immun 69, 6796-6803
19. Wilkinson, B. J., Sisson, S. P., Kim, Y., and Peterson, P. K. (1979) Infect Immun 26, 1159,-1163
20. Lee, L. Y., Hook, M, Haviland, D., Wetsel, R. A., Yonter, E. O., Syribeys, P., Vernachio, J., Brown, E. L. (2004) Journal of Infectious Diseases In Press
21. Boden, M. K., and Flock, J. I. (1994) Mol Microbiol 12,599-606
22. Boden Wastfelt, M. K., and Flock, J. I. (1995) J Clin Microbiol33, 2347-2352
23. Palma, M., Wade, D., Flock, M., and Flock, J. I. (1998) J Biol Chem 273, 13177-13181
24. Wade, D., Palma, M., Lofving-Arvholm, I., Sallberg, M., Silberring, J., and Flock, J. I. (1998) Biochem Biophys Res Commun 248,.690-695
25. Palma, M., Nozohoor, S., Schennings, T., Heimdahl, A., and Flock, J. I. (1996) Infect Immun 64, 5284-5289
26. Palma, M., Shannon, O., Quezada, H. C., Berg, A., and Flock, J. I. (₂₀₀₁) J Biol Chem 276, 31691-31697
27. Heilmann, C., Herrmann, M., Kehrel, B. E., and Peters, G. (2002) J Infect Dis 186, 32-39
28. Guo, B. P., Brown, E. L., Dorward, D. W., Rosenberg, L. C., and Höök, M. (1998) Mol Microbiol 30, 711-723
29. Lee, L. Y., Miyamoto, Y. J., McIntyre, B. W., Höök, M., McCrea, K. W., McDevitt, D., and Brown, E. L. (2002) J Clin Invest 110, 1461-1471.
30. Maniatis, T., Fritsch, E. F., and Sambrook, J. (1989) Molecular Cloning: A Laboratory Manual, Cold Spring Harbor Laboratory, Cold Spring Harbor, N.Y.
31. Brown, E. L., Guo, B. P., O'Neal, P., and Höök, M. (1999) J Bio Chem274, 26272-26278
32. Horstmann, R. D., Sievertsen, H. J., Knobloch, J., and Fischetti, V. A. (1988) Proc Natl Acad Sci USA 85, 1657-1661
33. Horstmann, R. D., Sievertsen, H. J., Leippe, M., and Fischetti, V. A. (1992) Infect Immun 60, 5036-5041,
34. Whitnack, E., and Beachey, E. H. (1985) J Exp Med 162, 1983-1997
35. Whitnack, E., and Beachey, E. H. (1985) J Bacteriol 164, 350-358
36. Murano, G. (1978) Am J Hematol 4, 409-417
37. Boackle, S. A. (2003) Biomed Pharmacother 57, 269-273
38. Kirschfink, M. (2001) Immunol Rev 180, 177-189

What is claimed is:

1. An isolated C3 binding region from the Staphylococcus aureus Efb protein having the ability to inhibit complement activation.

2. The C3 binding region of claim 1 wherein the binding region is located at the C-terminal end of the Efb protein.

3. The C3 binding region of claim 1 wherein the C3 binding region has an amino acid sequence of from amino acid 97 to 165 of the S. aureus Efb protein.

4. A pharmaceutical composition comprising the C3 binding region -of claim 1 and a pharmaceutically acceptable vehicle, carrier or excipient.

5. The pharmaceutical composition of claim 4 wherein the C3 binding region is present in an amount effective to inhibit complement activation.

6. An isolated antibody that recognizes the C3 binding region according to claim 1.

7. Isolated antisera containing the antibody according to claim 6.

8. A diagnostic kit comprising an antibody according to claim 6 and means for detecting binding by that antibody.

9. A diagnostic kit comprising the C3 binding region according to claim 1 and means for detecting binding to said protein fragment.

10. A method of diagnosing an infection of S. aureus comprising adding an antibody according to claim 6 to a sample suspected of being infected with S. aureus, and determining if antibodies have bound to the sample.

11. A pharmaceutical composition comprising the antibody of claim 6 and a pharmaceutically acceptable vehicle, carrier or excipient.

12. A method of inducing an immunological response comprising administering to a human or animal an immunogenic amount of an isolated C3 binding region according to claim 1.

13. An isolated nucleic acid coding for the C3 binding region according to claim 1.

14. The C3 binding region according to claim 1 which is produced by recombinant means.

15. A vaccine comprising the C3 binding region of claim 1 in an amount effective to elicit an immune response, and a pharmaceutically acceptable vehicle, carrier or excipient.

16. A method of inhibiting complement activity in a human or animal patient comprising administering to the patient the C3 binding region of claim 1 in an amount effective to inhibit complement activity.

17. The method of claim 16, wherein the C3 binding region is administered in the form of a pharmaceutical composition comprising the C3 binding region in an amount effective to inhibit complement activation and a pharmaceutically acceptable carrier.

18. A method of inhbiting complement activation in a human or animal patient in need of such inhibition comprising administering to the patent the Efb protein of Staphylococcus aureus or the C3 binding region of the Efb protein of Stahpylococcus aureus in an amount effective to inhibit complement activity.

19. The method of claim 18, wherein the Efb protein or C3 binding region is administered in the form of a pharmaceutical composition comprising the Efb protein or C3 binding region in an amount effective to inhibit complement activation and a pharmaceutically acceptable carrier.

20. A pharmaceutical composition comprising the S. aureus Efb protein or the C3 binding region of the S. aureus Efb protein in an amount effective to inhibit complement activation, and a pharmaceutically effective vehicle, carrier or excipient.

21. A method of treating or preventing hemolytic anemia in a human or animal patient in need of said treatment comprising administering to the patient the Efb protein of S. aureus or the C3-binding region of the Efb protein of S. aureus in an amount effective to inhibit complement activation.

22. The method of claim 21, wherein the Efb protein or the C3 binding region of the Efb protein is administered in the form of a pharmaceutical composition comprising the protein or binding region in an amount effective to inhibit complement activation and a pharmaceutically acceptable vehicle, carrier or excipient.

23. A method of reducing the induction of complement activation by a biological or prosthetic tissue or organ implant comprising coating the implant with an Efb protein or the C3 binding region of the Efb protein in an amount effective to inhibit complement activation when the implant is implanted into a human or animal patient.

24. A method of inducing an immunological response comprising administering to a patient an immunologically effective amount of the C3 binding region of the Staphylococcus epidermidis Efb protein.

25. The method of inhbiting complement activation according to claim 18 wherein said method is used to inhibit complement activation during a process intended to reduce the likelihood of rejection of a graft or implant.

26. The method of inhbiting complement activation according to claim 18 wherein said method is used to inhibit complement activation during a kidney dialysis process.

27. The method of inhbiting complement activation according to claim 26 wherein said method is used to inhibit complement activation during hemodialysis.


SEQUENCE LISTING (11 sequences)

SEQ ID NO: 1 - protein, 9 aa, Staphylococcus aureus
SEGYPREKK

SEQ ID NO: 2 - protein, 10 aa, Staphylococcus aureus
FTFEPFPTNE

SEQ ID NO: 3 - DNA, 34 bp, Staphylococcus aureus
cgcggatccccaagagaaaagaaaccagtgagta

SEQ ID NO: 4 - DNA, 30 bp, Staphylococcus aureus
aactgcagagttttatttaactaatccttg

SEQ ID NO: 5 - DNA, 30 bp, Staphylococcus aureus
cgcggatccccgtttcctacaaatgaagaa

SEQ ID NO: 6 - DNA, 27 bp, Staphylococcus aureus
aactgcagctagtatgcatattcatta

SEQ ID NO: 7 - DNA, 20 bp, Staphylococcus aureus
taatacgactcactataggg

SEQ ID NO: 8 - DNA, 21 bp, Staphylococcus aureus
ctagttattgctcagcggtgg

SEQ ID NO: 9 - DNA, 35 bp, Staphylococcus aureus
aactgcagttattctctcacaagattttgagcttg

SEQ ID NO: 10 - DNA, 24 bp, Staphylococcus aureus
ccagcagcgaaaactgatgcaact

SEQ ID NO: 11 - DNA, 30 bp, Staphylococcus aureus; identical to SEQ ID NO: 4